(19) United States
(12) Patent Application Publication
(10) Pub. No.: US 2023/0053658 A1
(43) Pub. Date: Feb. 23, 2023

(54) CIRCULATING NEDD9 IS INCREASED IN PULMONARY ARTERIAL HYPERTENSION

(71) Applicant: The Brigham and Women`s Hospital, Inc., Boston, MA (US)

(72) Inventors: Bradley A. Maron, Sharon, MA (US); Andriy Samokhin, Stoughton, MA (US); George A. Alba, Boston, MA (US)

(21) Appl. No.: 17/789,013

(22) PCT Filed: Dec. 23, 2020
(86) PCT No.: PCT/US20/66886

§ 371 Date: Jun. 24, 2022

Related U.S. Application Data
(60) Provisional application No. 62/953,902, filed on Dec. 26, 2019.

(51) Int. Cl. G01N 33/68; C12Q 1/6883

(52) U.S. Cl. CPC G01N 33/6893; C12Q 1/6883; C12Q 2600/158; G01N 2800/321

(57) ABSTRACT

Aspects of the present disclosure relate to methods for detecting neural precursor cell expressed developmentally down-regulated protein 9 (NEDD9) in a sample from a subject having or at risk for pulmonary hypertension (PH), wherein pulmonary hypertension (PH) is selected from the group consisting of pulmonary arterial hypertension (PAH), chronic thromboembolic pulmonary hypertension (CTEPH), and pulmonary hypertension (PH) due to acute respiratory distress syndrome (ARDS).

### CLAIM OF PRIORITY

This application claims the benefit of U.S. Provisional Patent Application No. 62/953,902, filed on Dec. 26, 2019, which is incorporated by reference herein in its entirety.

### FEDERALLY SPONSORED RESEARCH OR DEVELOPMENT

This invention was made with government support under Grant Nos. HL139613-01 and HL145420 awarded by the National Institutes of Health. The government has certain rights in the invention.

### FIELD OF THE INVENTION

The subject matter disclosed herein generally relates to methods of using neural precursor cell expressed developmentally down-regulated protein 9 (NEDD9) as a biomarker, e.g., as a biomarker for pulmonary hypertension (PH) such as pulmonary arterial hypertension (PAH).

### BACKGROUND OF THE INVENTION

Pulmonary arterial hypertension (PAH) is a severe cardiopulmonary disease defined, in part, by a fibrotic vasculopathy that increases pulmonary vascular resistance (PVR) and pathogenic remodeling of the right ventricle. Accumulating clinical trial data demonstrate a substantial therapeutic benefit in patients treated using an early-aggressive strategy. However, dyspnea and other non-specific complaints are often the first presenting symptom in PAH, contributing to delayed or missed diagnosis. In addition, longitudinal assessment of PAH progression generally hinges on right heart catheterization, echocardiography, or walk testing, which are invasive or limited by suboptimal precision. For these collective reasons, identifying a PAH-specific biomarker with diagnostic, prognostic relevance, and to guide therapeutic development has emerged as a major objective in the pulmonary vascular medicine field.

### SUMMARY OF THE INVENTION

The present disclosure is based, at least in part, on the finding that elevated levels of neural precursor cell expressed developmentally down-regulated protein 9 (NEDD9) can be found in plasma samples from patients having pulmonary hypertension (PH) (e.g., pulmonary arterial hypertension (PAH)) as compared to plasma samples from control patients (e.g., healthy patients).

Accordingly, aspects of the present disclosure provide a method for analyzing a sample, the method comprising providing a sample from a subject, and detecting neural precursor cell expressed developmentally down-regulated protein 9 (NEDD9) in the sample.

In some embodiments, detecting NEDD9 in the sample comprises detecting a level of NEDD9 protein in the sample. In some embodiment, the level of NEDD9 protein is detected by an immunoassay. In some embodiments, the immunoassay is an enzyme-linked immunosorbent assay (ELISA).

In some embodiments, detecting NEDD9 comprises detecting a level of NEDD9 nucleic acid.

In some embodiments, the sample is a plasma sample. In some embodiments, the sample is obtained from a subject having or suspected of having pulmonary hypertension (PH). In some embodiments, the pulmonary hypertension (PH) is selected from the group consisting of pulmonary arterial hypertension (PAH), chronic thromboembolic pulmonary hypertension (CTEPH), and pulmonary hypertension (PH) due to acute respiratory distress syndrome (ARDS).

In some embodiments, methods described herein further comprise administering the subject a treatment for pulmonary hypertension (PH).

In some embodiments, the subject is a human patient or a non-human animal.

In another aspect, a method is provided for diagnosing a subject as having pulmonary hypertension (PH), the method comprising providing a sample from the subject, detecting a level of NEDD9 in the sample, and comparing the level of NEDD9 in the sample to a reference level, wherein the presence of a level of NEDD9 in the sample that is above the reference level indicates that the subject has PH.

The details of one or more embodiments of the present disclosure are set forth in the description below. Other features or advantages of the present disclosure will be apparent from the following drawings and detailed description of several embodiments, and also from the appended claims.

### BRIEF DESCRIPTION OF THE DRAWINGS

FIGS. 1A-1F include LC-MS data confirming detection of NEDD9 in human plasma. Healthy control human plasma was immunoprecipitated with an anti-NEDD9 antibody covering amino acid sequences 82 to 398. In-gel trypsin digestion was performed on samples reduced with dithiothreitol before protein identification by in-tandem LC-MS. The MS1 spectra including the doubly charged y6, y7, or y8 ion corresponding to NEDD9 fragments is provided for peptides in the protein substrate domain (FIGS. 1A-1B), serine rich region (FIGS. 1C-1D), and C-terminal domain (FIG. 1E), illustrated graphically (FIG. 1F) (n=2 replicates per detected peptide, n=2 samples). Representative spectra are provided. LC-MS, liquid chromatography-mass spectrometry; m/z, mass-to-charge ratio.

FIGS. 2A-2E include data showing that plasma NEDD9 is increased significantly in PAH and PAH subtypes. Plasma was collected from 5 PAH referral centers, and the NEDD9 concentration for dyspnea non-PH controls (n=36) and patients with PAH (n=139) was quantified by ELISA (FIG. 1A). Compared with controls, plasma NEDD9 concentration was increased among patients with iPAH (N=54), CTD-PAH (n=52), CHD-PAH (n=7), porto-PAH (n=12), and toxin-induced—PAH (n=10) (FIG. 1B). Linear regression analyses correlating NEDD9 with mPAP (FIG. 1C) and TPG (FIG. 1D). Distribution of NEDD9 concentration by PVR quartile (FIG. 1E). For box plot analyses: mean, square; median, horizontal line; interquartile range, box distribution; maximum and minimum, y-axis lines. CHD, congenital heart disease; CTD, connective tissue disease; ELISA, enzyme-linked immunosorbent assay; iPAH, idiopathic pulmonary arterial hypertension; mPAP, mean pulmonary artery pressure; PAH, pulmonary arterial hypertension; PH, pulmonary hypertension; porto-PAH, portopulmonary hypertension; PVR, pulmonary vascular resistance; TPG, transpulmonary gradient.

FIGS. 3A-3C include data showing the association between plasma NEDD9 and cardiopulmonary hemodynamic measurements in iPAH. Plasma was collected from 5 PAH referral centers and the NEDD9 concentration for dyspnea non-PH controls (n=36) and patients with iPAH (n=54) was quantified by ELISA. These data were correlated with mPAP (FIG. 3A), TPG (FIG. 3B), and PVR (FIG. 3C). ELISA, enzyme-linked immunosorbent assay; iPAH, idiopathic pulmonary arterial hypertension; mPAP, mean pulmonary artery pressure; PAH, pulmonary arterial hypertension; PH, pulmonary hypertension; PVR, pulmonary vascular resistance; TPG, transpulmonary gradient.

FIGS. 4A-4C includes data showing the association between plasma NEDD9 and cardiopulmonary hemodynamic measurements in CTD-PAH. Plasma was collected from 5 PAH referral centers and the NEDD9 concentration for dyspnea non-PH controls (n=36) and patients with CTD-PAH (n=53) was quantified by ELISA. These data were correlated with mPAP (FIG. 4A), TPG (FIG. 4B), and PVR (FIG. 4C). CTD, connective tissue disease; ELISA, enzyme-linked immunosorbent assay; mPAP, mean pulmonary artery pressure; PAH, pulmonary arterial hypertension; PH, pulmonary hypertension; PVR, pulmonary vascular resistance; TPG, transpulmonary gradient.

FIGS. 5A-5C includes data showing the association between plasma NEDD9 and cardiopulmonary hemodynamic measurements in CHD-PAH. Plasma was collected from 5 PAH referral centers and the NEDD9 concentration for dyspnea non-PH controls (n=36) and patients with CTD-PAH (n=10) was quantified by ELISA. These data were correlated with mPAP (FIG. 5A), TPG (FIG. 5B), and PVR (FIG. 5C). CHD, congenital heart disease; ELISA, enzyme-linked immunosorbent assay; mPAP, mean pulmonary artery pressure; PAH, pulmonary arterial hypertension; PH, pulmonary hypertension; PVR, pulmonary vascular resistance; TPG, transpulmonary gradient.

FIGS. 6A-6E include data showing the association between plasma NEDD9 and alternative hemodynamic prognostic measurements at rest and during exercise. From the overall cohort, adjustment for analyses for age, sex, and PAH subtype identified a significant relationship between NEDD9 and PVR among patients in the Partners A and JHU cohorts. In subsequent subgroup analyses focusing on patients from these centers, NEDD9 correlated inversely with resting MvO₂ (FIG. 6A) (n=19), CO at peak exercise (FIG. 6B), change in CO from rest to peak exercise (FIG. 6C), PVR at peak exercise on invasive cardiopulmonary exercise testing (FIG. 6D), and RVEF at rest assessed by cardiac magnetic resonance imaging (FIG. 6E) (n=45 for FIGS. 6B-6E). CO, cardiac output; JHU, Johns Hopkins University; MvO₂, mixed venous oxygen saturation level; PAH, pulmonary arterial hypertension; PVR, pulmonary vascular resistance; RVEF, right ventricular ejection fraction.

FIGS. 7A-7B include data showing that plasma NEDD9 predicts PAH diagnosis and is associated with adverse clinical events. Receiver operating characteristic curve showing the strength of plasma NEDD9 for predicting pulmonary arterial hypertension (PAH) diagnosis (FIG. 7A). Time to event plot for unadjusted lung transplantation and mortality-free survival for patients with PAH stratified by the median plasma NEDD9 level from the study cohort (log-rank test, X²=5.14; P=0.023) (FIG. 7B).

FIGS. 8A-8E include data showing that plasma NEDD9 concentration measured by ELISA correlates strongly with concentration analyzed by immunoblot. Representative standard curve for the commercially purchased NEDD9 ELISA (FIG. 8A). Anti-NEDD9 immunoblot (Abcam, catalogue #ab18056) was performed on the ELISA standard to verify NEDD9 expression (S, sample) (FIG. 8B). NEDD9 expressed in E. coli cells from Origene and confirmed by anti-NEDD9 immunoblot was not detected by the NEDD9 ELISA (N=3) (ND, not detectable) (FIG. 8C). Anti-NEDD9 immunoprecipitation (IP) was performed on lysates from cultured human pulmonary artery endothelial cells (HPAECs) and loaded onto the NEDD9 ELISA at different dilutions (FIG. 8D). In contrast to findings using recombinant NEDD9 (FIG. 8C), ELISA performed using NEDD9-IP samples from HPAECs resulted in a detectable concentration of NEDD9 (FIG. 8D). Correlation between plasma NEDD9 concentration measured using anti-NEDD9 immunoblot and NEDD9 ELISA (N=7 PAH patients) (FIG. 8E). ELISA, enzyme-linked immunosorbent assay; PAH, pulmonary arterial hypertension.

FIGS. 9A-9E include schematic depictions of the sample throughput for each center participating in this study. Partners A is Brigham and Women's Hospital (FIG. 9A), Partners B is Massachusetts General Hospital (FIG. 9B), Rhode Island Hospital/Brown University (FIG. 9C), University of Washington (Seattle) (FIG. 9D), The Johns Hopkins University (FIG. 9E). iPAH, idiopathic pulmonary arterial hypertension; WHO, World Health Organization; PH, pulmonary hypertension; CTD, connective tissue disease; CHD, congenital heart disease; porto-PAH, portopulmonary hypertension; SSc, Systemic sclerosis; ILD, interstitial lung disease; LHD, left heart disease. Color coding across images is according to disease prevalence.

FIGS. 10A-10C include graphs showing center-specific differences in patient plasma NEDD9 concentration. The plasma NEDD9 level was quantified by ELISA in samples from dyspnea non-PH controls and idiopathic pulmonary arterial hypertension (iPAH) patients referred to one of three or five PAH referral centers, respectively. Significant differences in NEDD9 level were observed across controls at Partners A (N=9), Partners B (N=24), and the Johns Hopkins University (JHU) (N=3) (FIG. 10A), as well as across the iPAH cohorts at Partners A (N=10), Partners B (N=11), Rhode Island Hospital (RIH)/Brown University (N=6), University of Washington (UW) (N=17), and JHU (N=10) inclusive of all controls (N=36) (FIG. 10B). P<0.0001 by ANOVA. In the Partners B cohort, no significant difference was observed in NEDD9 concentration between controls (N=24) vs. PAH (WHO Group 1 PH) (N=31), WHO Group 2 PH (N=15), WHO Group 3 (N=12), PH mixed etiology (N=17), and exercise-induced PH (N=3) (FIG. 10C). For box plot analyses: mean, square; median, horizontal line; interquartile range, box distribution; maximum and minimum, y-axis lines.

The details of one or more embodiments of the invention are set forth in the description below. Other features or advantages of the present invention will be apparent from the following drawings and detailed description of several embodiments, and also from the appended claims.

### DETAILED DESCRIPTION

Aspects of the present disclosure relate to methods for detecting neural precursor cell expressed developmentally down-regulated protein 9 (NEDD9) in a sample (e.g., a plasma sample) from a subject (e.g., a patient) having or at risk for pulmonary hypertension (PH), e.g., pulmonary arterial hypertension (PAH).

Such methods can be useful for clinical purposes, e.g., identifying a subject having or at risk for pulmonary hypertension, selecting a treatment, monitoring pulmonary hypertension progression, assessing the efficacy of a treatment against pulmonary hypertension, or determining a course of treatment for a subject.

The assay methods described herein can also be useful for non-clinical applications, e.g., for research purposes, including, e.g., studying the mechanism of pulmonary hypertension development and/or biological pathways and/or biological processes involved in pulmonary hypertension, and developing new therapies for pulmonary hypertension based on such studies.

Methods described herein are based, at least in part, on the identification of NEDD9 as a biomarker for pulmonary hypertension (PH), e.g., pulmonary arterial hypertension (PAH). As used herein, the term “biomarker” refers to a biological molecule that is present at a level in a subject that deviates from a level of the same biological molecule in a different subject. For example, NEDD9 that is indicative of pulmonary hypertension (PH) can have an elevated level in a sample from a subject (e.g., a plasma sample from a subject that has or is at risk for pulmonary hypertension) relative to the level of NEDD9 in a control sample (e.g., a plasma sample from a healthy subject such as a subject who does not have or is not at risk for pulmonary hypertension).

NEDD9 is a docking protein that plays a central coordinating role for tyrosine-kinase-based signaling related to cell adhesion. NEDD9 is implicated in the pathogenesis of various solid tumor cancers. For example, in breast adenocarcinoma, NEDD9 targets transforming growth factor-β to alter the phenotype of cells and permit blood-borne metastasis. The amino acid sequence of human NEDD9 is provided in UniProtKB accession number Q14511.

Pulmonary hypertension (PH) is defined as mean pulmonary artery pressure >20 mm Hg. Pulmonary hypertension has an estimated prevalence of 10-20% within the general population. The World Health Organization (WHO) categorizes pulmonary hypertension into five groups based on etiologies resulting in similar histopathologic changes. These groups comprise pulmonary arterial hypertension (PAH), pulmonary venous hypertension (PVH) due to left heart disease, pulmonary hypertension (HTN) due to lung disease/hypoxemia, chronic thromboembolic pulmonary hypertension (CTEPH), and pulmonary hypertension due to unclear multifactorial mechanisms.

Pulmonary hypertension (PH) includes pulmonary hypertension due to a disease such as acute respiratory distress syndrome (ARDS), scleroderma patients, sickle cell anemia, HIV, mixed connective-tissue disease, congenital heart disease (CHD), chronic obstructive pulmonary disease (COPD), hereditary hemorrhagic telangiectasia (HHT), sleep apnea, liver disease, or lupus.

Experimental data provided herein shows for the first time that circulating levels of the pro-fibrotic protein NEDD9 are increased significantly in PAH and correlate with key cardiopulmonary hemodynamic and right ventricular parameters that inform prognosis. This effect was maintained despite center-specific differences in control and PAH NEDD9 levels, and wide heterogeneity in the clinical profile of the study population for age, sex, and other important clinical covariates. Additionally, NEDD9 associated inversely with left atrial hypertension induced by exercise. This was directionally opposite from NT-proBNP, which is a non-specific indicator of left- or right-sided myocardial stretch, implying that in this study NEDD9 was not coupled to left atrial hypertension. Further, increased plasma NEDD9 was associated with elevated risk for lung transplantation or mortality. Collectively, these data identify NEDD9 as a potential biomarker that provides important insight on pulmonary vascular-right ventricular pathophysiology and clinical outcome in PAH.

Biomarker discovery has emerged as a major objective in the PAH field owing to delayed and inaccurate diagnosis that is reported commonly by referral centers, and because the optimal strategy for monitoring disease progression requires invasive testing. To this end, recent proteomic analyses leveraging multiplex platforms have reported novel independent predictors of adverse outcome, including many proteins unrecognized previously in the pathogenesis of PAH. Others have reported hypothesis-driven investigations in which the pathophysiological relevance of a putative biomarker was based on data from cardiovascular diseases with overlapping, but not distinct, pathophysiology compared to PAH. These include osteopontin, C-reactive protein, Galectin-3, among others, as well as troponin-T and NT-proBNP that are used commonly in clinical practice today. Studies described herein diverge from these prior studies by showing that NEDD9, a protein with specific pathobiological relevance to fibrotic and hypertrophic vascular remodeling in PAH, is also relevant to PAH patients clinically.

The magnitude of increase in circulating NEDD9 in PAH compared to controls, predictive value of NEDD for diagnosing PAH, and the strength of association between NEDD9 and selected hemodynamic predictors of PAH clinical events in studies described herein was equivalent or, in some instances, superior to established biomarkers. For example, although a strong association between NT-proBNP and PVR was reported in one study involving a small cohort of iPAH patients, subsequent data from cross-sectional or unselected populations suggests that this relationship is comparatively weaker than that of NEDD9 and PVR described herein. In addition, the relationship between NEDD9 and mPAP, PVR, and transpulmonary gradient was increased when focusing the instant analysis on circumspect PAH subtypes, suggesting that pathophysiological or clinical heterogeneity may have contributed to differing results from analyses involving the total PAH population. This observation, in turn, underscores the importance of optimal phenotyping when linking biochemical measurements to patient profile, and suggests that additional work on NEDD9 in PAH clinically should consider diseases subtypes carefully.

As described herein, rising plasma NEDD9 levels were associated with a significant increase in the adjusted risk for mortality or lung transplantation. Based on studies described herein, NEDD9 can be linked to PAH arterial remodeling, abnormal cardiopulmonary hemodynamics, and adverse clinical events. Therefore, a strong framework has been established for NEDD9 as a potentially useful, measurable, and informative indicator of PAH severity relative to pathobiology, pathophysiology and clinical risk. Experimental data reported herein supports using plasma NEDD9 concentration as a metric for enrollment in clinical trials studying emergent therapies that aim to inhibit NEDD9 in PAH and other diseases of similar pathobiology, and suggest that additional studies dedicated to determining the prognostic utility of plasma NEDD9 within the context of patient care are warranted.

A key strength of this work relates to the use of multiple methodologies to verify NEDD9 detection in human plasma, including definitive peptide identification by LC-MS. Results from these experiments imply that full length NEDD9 is present and potentially biologically active in circulation. Thus, although increased NEDD9 is observed from peripheral blood samples in patients with gastric adenocarcinoma and other cancers, the results described herein provide additional information that may refine biomarker studies in the future through quantitative proteomic analysis to determine the optimal NEDD9 fragment corresponding to PAH diagnosis, prognosis, and treatment response, for example.

Pulmonary endothelial NEDD9 is a critical mediator of vascular fibrosis in PAH. It is therefore noteworthy that in CTD-PAH, which is characterized mainly by a fibrotic arteriopathy, NEDD9 was particularly elevated and associated with cardiopulmonary hemodynamics as well as 6-minute walk distance. It is not possible to know from experimental data provided herein if pulmonary endothelial and plasma NEDD9 concentration in any PAH subtype are related quantitatively or mechanistically. However, NEDD9 is reported to translocate to blood in metastatic processes, and transcellular signaling involving pulmonary endothelial NEDD9 has been detailed previously. Paracrine and endocrine biofunctionality of microRNAs, hormones, and vasoactive peptides are recognized increasingly in pulmonary and systemic manifestations of PAH. Findings described herein provide further evidence in support of dedicated studies tracing the afferent and efferent arc of such disease mediators detected systematically.

The mechanisms regulating metabolism of cell-free NEDD9 have not been elucidated. Experimental data provided herein showed that NEDD9 concentration did not appear to be dependent on renal dysfunction or hemoglobin content, which might have provided insight on its bioelimination. Similarly, an association was not observed between resting CO and NEDD9, implying that regulation of NEDD9 is likely uncoupled from typical neurohumoral signaling pathways in PAH such as the renin-angiotensin axis. By contrast, findings described herein suggest that NEDD9 regulation may be a pulmonary vascular-right ventricular process, since exercise PAWP nor resting PAWP in WHO Group 2 PH patients (both indicative of elevated LV or left atrial pressure) associated with plasma NEDD9, whereas such an effect was observed between NEDD9 and selected right ventricular-specific measurements.

Described herein is a retrospective analysis that included a combination of incident and prevalent PAH patients, is subject to referral and selection bias, and appears to include batch effects relative to center participation, which, in turn, could have been due to differences in vascular sampling site, changes in protein stability over the study period, or other untested or confounding variables. This was particularly the case for controls, which were not available from all participating centers. Despite improvement in availability of clinical samples through the effort of academic initiatives worldwide, PAH is, nonetheless, a rare disease making adherence to standardized and tightly controlled collection protocols across institutions challenging. This may have affected outcome data in the study described herein, which included patients across a variable and wide follow-up time interval. Similarly, heterogeneity in the diagnostic approach to PAH patients by center and region and may have affected the uniformity of the catheterization (and other clinical) data included in the study described. Therefore, additional prospective data are needed to fully characterize the utility of NEDD9 as a bona fide PAH biomarker.

Taken together, in a multi-center, retrospective observational study described herein, plasma NEDD9 was increased significantly in PAH. This effect was maintained across five PAH subtypes, but elevated NEDD9 was not observed in other forms of PH. Plasma NEDD9 was strongly predictive of PAH diagnosis, correlated with established hemodynamic and right ventricular parameters, and associated with increased hard clinical events.

### I. Methods for Detection of NEDD9

Any sample that may contain NEDD9 can be analyzed by the assay methods described herein. The methods described herein involve providing a sample obtained from a subject. In some examples, the sample may be from an in vitro assay, e.g., from an in vitro cell culture (e.g., an in vitro cell culture of pulmonary artery endothelial cells). In other examples, the sample to be analyzed by the assay methods described herein is a biological sample.

As used herein, a “sample” refers to a composition that comprises biological materials including, but not limited to, plasma, tissue, cells, and/or fluid from a subject. A sample includes both an initial unprocessed sample taken from a subject as well as subsequently processed, e.g., partially purified or preserved forms. In some embodiments, the sample is plasma. In some embodiments, multiple (e.g., at least 2, 3, 4, 5, or more) samples may be collected from a subject, over time or at particular time intervals, for example, to assess the disease progression or evaluate the efficacy of a treatment. A sample can be obtained from a subject using any means known in the art.

The term “subject” or “patient” can be used interchangeably and refers to a subject who needs the analysis as described herein. In some embodiments, the subject is a human or a non-human mammal (e.g., cat, dog, horse, cow, goat, or sheep). In some embodiments, a subject is suspected of or is at risk for pulmonary hypertension (PH), e.g., pulmonary arterial hypertension (PAH), chronic thromboembolic pulmonary hypertension (CTEPH), or pulmonary hypertension due to acute respiratory distress syndrome (ARDS). Such a subject can exhibit one or more symptoms associated with pulmonary hypertension. Alternatively or in addition to, such a subject can have one or more risk factors for pulmonary hypertension, e.g., a family history of pulmonary hypertension.

Alternatively, the subject who needs the analysis described herein can be a patient having pulmonary hypertension. Such a subject can currently be having a relapse, or can have suffered from the disease in the past (e.g., currently relapse-free). In some examples, the subject is a human patient who can be on a treatment of the disease, e.g., a treatment involving an anti-hypertensive agent. In other instances, such a human patient can be free of such a treatment.

Examples of pulmonary hypertension (PH) include, without limitation, pulmonary arterial hypertension (PAH), pulmonary hypertension due to left heart disease, pulmonary hypertension due to lung disease, chronic thromboembolic pulmonary hypertension (CTEPH), pulmonary hypertension with unclear or multifactorial mechanisms, or pulmonary hypertension due to acute respiratory distress syndrome (ARDS).

Pulmonary arterial hypertension (PAH) includes various subtypes, e.g., idiopathic PAH (iPAH), portopulmonary hypertension, human immunodeficiency virus-PAH, pulmonary veno-occlusive disease, exercise-induced PH, drug-induced-PAH, and toxin-induced-PAH.

Any sample (e.g., those described herein) can be used in the methods described herein, which involve measuring the level of NEDD9 as described herein. Levels (e.g., the amount) of NEDD9 disclosed herein, or changes in levels of NEDD9, can be assessed using conventional assays or those described herein.

As used herein, the terms “measuring” or “measurement,” or alternatively “detecting” or “detection,” means assessing the presence, absence, quantity or amount (which can be an effective amount) of NEDD9 within a sample, including the derivation of qualitative or quantitative concentration levels of NEDD9, or otherwise evaluating the values and/or categorization of NEDD9 in a sample from a subject.

In some embodiments, the level of NEDD9 is assessed or measured by directly detecting NEDD9 protein in a sample (e.g., a plasma sample). Alternatively, or in addition to, the level of NEDD9 protein can be assessed or measured indirectly in a sample, for example, by detecting the level of activity of NEDD9 protein.

The level of NEDD9 protein can be measured using an immunoassay. Examples of immunoassays include any known assay (without limitation), and can include any of the following: immunoblotting assay (e.g., Western blot), immunohistochemical analysis, flow cytometry assay, immunofluorescence assay (IF), enzyme-linked immunosorbent assays (ELISAs) (e.g., sandwich ELISAs), radioimmunoassays, electrochemiluminescence-based detection assays, magnetic immunoassays, lateral flow assays, and related techniques. Additional suitable immunoassays for detecting NEDD9 protein will be apparent to those of skill in the art.

Such immunoassays can involve the use of an agent (e.g., an antibody) specific to NEDD9. An agent such as an antibody that “specifically binds” to NEDD9 is a term well understood in the art, and methods to determine such specific binding are also well known in the art. An antibody is said to exhibit “specific binding” if it reacts or associates more frequently, more rapidly, with greater duration and/or with greater affinity with NEDD9 than it does with other proteins. It is also understood by reading this definition that, for example, an antibody that specifically binds to a first target peptide may or may not specifically or preferentially bind to a second target peptide. As such, “specific binding” or “preferential binding” does not necessarily require (although it can include) exclusive binding Generally, but not necessarily, reference to binding means preferential binding. In some examples, an antibody that “specifically binds” to a target peptide or an epitope thereof may not bind to other peptides or other epitopes in the same antigen.

As used herein, the term “antibody” refers to a protein that includes at least one immunoglobulin variable domain or immunoglobulin variable domain sequence. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and domain antibodies (dAb) fragments (de Wildt et al., Eur J Immunol. 1996; 26(3):629-39)) as well as complete antibodies. An antibody can have the structural features of IgA, IgG, IgE, IgD, IgM (as well as subtypes thereof). Antibodies may be from any source including, but not limited to, primate (human and non-human primate) and primatized (such as humanized) antibodies.

In some embodiments, the antibodies as described herein can be conjugated to a detectable label and the binding of the detection reagent to NEDD9 can be determined based on the intensity of the signal released from the detectable label. Alternatively, a secondary antibody specific to the detection reagent can be used. One or more antibodies may be coupled to a detectable label. Any suitable label known in the art can be used in the assay methods described herein. In some embodiments, a detectable label comprises a fluorophore. As used herein, the term “fluorophore” (also referred to as “fluorescent label” or “fluorescent dye”) refers to moieties that absorb light energy at a defined excitation wavelength and emit light energy at a different wavelength. In some embodiments, a detection moiety is or comprises an enzyme. In some embodiments, an enzyme is one (e.g., β-galactosidase) that produces a colored product from a colorless substrate.

It should be understood that more than one suitable anti-NEDD9 antibody can be used in methods described herein, for example, those known in the art or described herein. Anti-NEDD9 antibodies can be found in, e.g., International Application No. PCT/US2019/059890, filed Nov. 5, 2019, which published as WO2020097096, the relevant disclosure of the prior application is herein incorporated by reference for the purposes and subject matter referenced herein.

Examples of anti-NEDD9 antibodies include, but are not limited to, mouse monoclonal antibody [2G9] (Abcam), mouse monoclonal antibody [1B4] (Abnova), mouse monoclonal antibody [14A11] (Invitrogen), and rabbit polyclonal antibodyABT166 (Millipore Sigma).

In some examples, an assay method described herein is applied to measure the level of NEDD9 in a sample, which can be a blood sample or a plasma sample. Any of the assays known in the art, e.g., immunoassays can be used for measuring the level of NEDD9.

It will be apparent to those of skill in the art that this disclosure is not limited to immunoassays. Detection assays that are not based on an antibody, such as mass spectrometry, are also useful for the detection and/or quantification of NEDD9. Assays that rely on a chromogenic substrate can also be useful for the detection and/or quantification of NEDD9.

Alternatively, the level of nucleic acids encoding NEDD9 in a sample can be measured via a conventional method. In some embodiments, measuring the expression level of nucleic acid encoding NEDD9 comprises measuring mRNA. In some embodiments, the expression level of mRNA encoding NEDD9 can be measured using real-time reverse transcriptase (RT) Q-PCR or a nucleic acid microarray. Methods to detect biomarker nucleic acid sequences include, but are not limited to, polymerase chain reaction (PCR), reverse transcriptase-PCR (RT-PCR), in situ PCR, quantitative PCR (Q-PCR), real-time quantitative PCR (RT Q-PCR), in situ hybridization, Southern blot, Northern blot, sequence analysis, microarray analysis, detection of a reporter gene, or other DNA/RNA hybridization platforms.

Any binding agent that specifically binds to NEDD9 may be used in methods described herein to measure the level of NEDD9 in a sample. In some embodiments, the binding agent is an antibody or an aptamer that specifically binds to NEDD9 protein. In other embodiments, the binding agent may be one or more oligonucleotides complementary to NEDD9 nucleic acid.

To measure the level of a target biomarker, a sample can be in contact with a binding agent under suitable conditions. In general, the term “contact” refers to an exposure of the binding agent with the sample or cells collected therefrom for suitable period sufficient for the formation of complexes between the binding agent and NEDD9 (e.g., nucleic acid or protein) in the sample, if any. In some embodiments, the contacting is performed by capillary action in which a sample is moved across a surface of the support membrane.

In some embodiments, the assays can be performed on low-throughput platforms, including single assay format. For example, a low-throughput platform can be used to measure the presence and amount of NEDD9 protein in a sample (e.g., a plasma sample) for diagnostic methods, monitoring of disease and/or treatment progression, and/or predicting whether a disease or disorder may benefit from a particular treatment.

In some embodiments, it may be necessary to immobilize a binding agent to the support member. Methods for immobilizing a binding agent will depend on factors such as the nature of the binding agent and the material of the support member and may require particular buffers. Such methods will be evident to one of ordinary skill in the art. For example, NEDD9 in a sample can be measured using any method described herein.

The type of detection assay used for the detection and/or quantification of NEDD9 may depend on the particular situation in which the assay is to be used (e.g., clinical or research applications), on the kind of NEDD9 to be detected (e.g., nucleic acid or protein), and/or on the kind and number of patient samples to be run in parallel, to name a few parameters.

The assay methods described herein may be used for both clinical and non-clinical purposes. Some examples are provided herein.

### II. Application of Detection of NEDD9

Methods described herein can be applied for evaluation of disease, e.g., diagnosis or prognosis of a disease. Evaluation can include identifying a subject as being at risk for or having a disease as described herein, e.g., pulmonary hypertension (PH), pulmonary arterial hypertension (PAH), pulmonary hypertension due to left heart disease, pulmonary hypertension due to lung disease, chronic thromboembolic pulmonary hypertension (CTEPH), pulmonary hypertension with unclear or multifactorial mechanisms, or pulmonary hypertension due to acute respiratory distress syndrome (ARDS). Evaluation can also include monitoring treatment of a disease, such as evaluating the effectiveness of a treatment for pulmonary hypertension.

(a) Diagnosis or Prognosis

In some embodiments, the methods described herein are used to determine the level of NEDD9 in a sample (e.g., a plasma sample) collected from a subject (e.g., a human patient suspected of having pulmonary hypertension). The NEDD9 level is then compared to a reference value to determine whether the subject has or is at risk for pulmonary hypertension. The reference value can be a control level of NEDD9. In some embodiments, the control level is a level of NEDD9 in a control sample. In some embodiments, a control sample is obtained from a healthy subject or population of healthy subjects. As used herein, a healthy subject is a subject that is apparently free of pulmonary hypertension at the time the level of NEDD9 is measured or has no history of pulmonary hypertension.

In some embodiments, the amount by which the level (or score) in the subject is less than the reference level (or score) is sufficient to distinguish a subject from a control subject, and optionally is a statistically significantly less than the level (or score) in a control subject. In cases where the level (or score) of NEDD9 in a subject being equal to the reference level (or score) of NEDD9, the “being equal” refers to being approximately equal (e.g., not statistically different).

Suitable reference values can be determined using methods known in the art, e.g., using standard clinical trial methodology and statistical analysis. The reference values can have any relevant form. In some cases, the reference comprises a predetermined value for a meaningful score or level of NEDD9, e.g., a control reference level that represents a normal level of NEDD9, e.g., a level in an unaffected subject or a subject who is not at risk of developing pulmonary hypertension (PH), and/or a disease reference that represents a level of NEDD9 associated with risk of developing pulmonary hypertension (PH).

The predetermined level or score can be a single cut-off (threshold) value, such as a median or mean, or a level or score that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level or score is a level or score determined in the same subject, e.g., at a different time point, e.g., an earlier time point.

The control level can also be a predetermined level. Such a predetermined level can represent the level of NEDD9 in a population of subjects that do not have or are not at risk for pulmonary hypertension. The predetermined level can take a variety of forms. For example, it can be a single cut-off value, such as a median or mean. In some embodiments, such a predetermined level can be established based upon comparative groups, such as where one defined group is known to have pulmonary hypertension and another defined group is known to not have pulmonary hypertension. Alternatively, the predetermined level can be a range, for example, a range representing the levels of NEDD9 in a control population within a predetermined percentile.

The control level as described herein can be determined by various methods. In some embodiments, the control level can be obtained by performing a known method. In some embodiments, the control level can be obtained by performing the same assay used for determine the level of NEDD9 in a sample from a subject. In some embodiments, the control level can be obtained by performing a method described herein. In some embodiments, the control level can be obtained from members of a control population and the results can be analyzed by, e.g., a computational program, to obtain the control level (a predetermined level) that represents the level of NEDD9 in the control population.

By comparing the level of NEDD9 in a sample obtained from a subject to the reference value as described herein, it can be determined as to whether the subject has or is at risk for pulmonary hypertension. For example, if the level of NEDD9 of the subject is elevated from the reference value (e.g., increased as compared to the reference value), the candidate subject might be identified as having or at risk for pulmonary hypertension (e.g., PAH). In another example, if the level of NEDD9 in a plasma sample from a subject elevated from the reference value (e.g., increased as compared to the reference value, e.g., 12 ng/ml), the candidate subject might be identified as having or at risk for mortality and/or as being in need of a transplant and/or as being in need of treatment for pulmonary hypertension (PH).

As used herein, “an elevated level or a level above a reference value” means that the level of NEDD9 is higher than a reference value, such as a predetermined threshold or a level of NEDD9 in a control sample.

An elevated level of NEDD9 includes a NEDD9 level that is, for example, 1%, 5%, 10%, 20%, 30%, 40%, 50%, 60%, 70%, 80%, 90%, 100%, 150%, 200%, 300%, 400%, 500% or more above a reference value. An elevated level of NEDD9 also includes increasing a phenomenon from a zero state (e.g., no or undetectable NEDD9 in a sample) to a non-zero state (e.g., some or detectable NEDD9 in a sample).

As used herein, “a decreased level or a level below a reference value” means that the level of NEDD9 is lower than a reference value, such as a predetermined threshold or a level of NEDD9 in a control sample.

An decreased level of NEDD9 includes a NEDD9 level that is, for example, 1%, 5%, 10%, 20%, 30%, 40%, 50%, 60%, 70%, 80%, 90%, 100%, 150%, 200%, 300%, 400%, 500% or more below a reference value. A decreased level of NEDD9 also includes decreasing a phenomenon from a non-zero state (e.g., some or detectable NEDD9 in a sample) to a zero state (e.g., no or undetectable NEDD9 in a sample).

In some embodiments, the subject is a human patient having a symptom of pulmonary hypertension, e.g., those disclosed herein such as PAH. For example, the subject has dyspnea, fatigue, syncope, chest pain, edema, cyanosis, heart palpitations, or a combination thereof. In some embodiments, the subject has no symptom of pulmonary hypertension at the time the sample is collected, has no history of a symptom of pulmonary hypertension, or no history of pulmonary hypertension.

In some embodiments, the subject has pulmonary hypertension due to a disease such as acute respiratory distress syndrome (ARDS), scleroderma patients, sickle cell anemia, HIV, mixed connective-tissue disease, congenital heart disease (CHD), chronic obstructive pulmonary disease (COPD), hereditary hemorrhagic telangiectasia (HHT), sleep apnea, liver disease, lupus, or other diseases evident to those skilled in the art as causing pulmonary hypertension (PH).

In some embodiments, the subject is resistant to a treatment such as an anti-hypertensive agent.

(b) Evaluation of Treatment Effectiveness

Methods described herein can also be applied to evaluate the effectiveness of a treatment for pulmonary hypertension (e.g., PAH). For example, multiple samples (e.g., plasma samples) can be collected from a subject to whom a treatment is performed either before and after the treatment or during the course of the treatment. The levels of NEDD9 can be measured by any method described herein. If the level of NEDD9 decrease after the treatment or over the course of the treatment (the level of NEDD9 in a later collected sample as compared to that in an earlier collected sample), remains the same or decrease, it indicates that the treatment is effective.

In the subject is identified as not responsive to the treatment, a higher dose and/or frequency of dosage of the therapy can be administered to the subject identified. In some embodiments, the dosage or frequency of dosage of the therapy is maintained, lowered, or ceased in a subject identified as responsive to the treatment or not in need of further treatment. Alternatively, a different treatment can be applied to the subject who is found as not responsive to the first treatment.

(c) Non-Clinical Applications

Methods described herein can also be applied to non-clinical uses, e.g., for research purposes. For example, methods described herein can be used to identify novel biological pathways or processes involved in pulmonary hypertension (e.g., pulmonary arterial hypertension).

In some embodiments, methods described herein can be applied to the development of a new therapy. For example, the levels of NEDD9 can be measured in samples obtained from a subject having been administered a new therapy (e.g., in a clinical trial). In some embodiments, the level of NEDD9 can indicate the efficacy of the new therapy or the progress of pulmonary hypertension in the subject prior to, during, or after the new therapy.

### III. Treatment of Pulmonary Hypertension (PH)

A subject having or at risk for pulmonary hypertension (PH) (e.g., pulmonary arterial hypertension (PAH)), as identified using the methods described herein, may be treated with any appropriate therapy. Non-limiting examples of a therapy for use in methods described herein include an anti-hypertensive agent, an anticoagulant, oxygen therapy, a surgery, or a NEDD9 antagonist (e.g., an anti-NEDD9 antibody, a small molecule inhibitor of NEDD9, a peptide inhibitor of NEDD9, or an agent that inhibits expression of NEDD9 such as an interfering RNA that targets NEDD9). In some embodiments, the therapy can target a molecule in a NEDD9 pathway, e.g., the therapy can be an endothelin receptor antagonist such as macitentan, bosentan, and ambrisentan. A therapy also encompasses a life style change, e.g., a low-salt diet and/or exercise.

In some embodiments, methods provided herein include selecting a treatment for a subject based on the output of the described method, e.g., measuring the level of neural precursor cell expressed developmentally down-regulated protein 9 (NEDD-9). Alternatively, or in addition to, methods provided herein include administering a treatment for a subject based on the output of the described method.

In some embodiments, the therapy comprises administering an antihypertensive agent. Examples of anti-hypertensive agents include, but are not limited to, angiotensin converting enzyme inhibitors (ACEi) (e.g., benazepril, fosinopril, lisinopril), centrally α2-adrenergic agonists (e.g., methyldopa, clonidine), peripherally acting adrenergic-receptor antagonists (e.g., labetalol, prazosin), calcium channel blockers (e.g., amlodipine, diltiazem, nifedipine), vasodilators (e.g., hydralazine, sodium nitroprusside, epoprostenol, treprostinil), and diuretics (e.g., thiazide diuretics such as chlorothiazide, chlorthalidone, hydrochlorothiazide, indapamide, and metolazone).

In some embodiments, the therapy comprises administering an anticoagulant. Examples of anticoagulants include, but are not limited to, glycoprotein platelet inhibitors (e.g., abciximab, eptifibatide, tirofiban), platelet aggregation inhibitors (e.g., aspirin, cangrelor, cilostazol, clopidogrel, dipyridamole, prasugrel, ticlopidine, ticagrelor) and protease-activated receptor-1 antagonists (e.g., vorapaxar).

In some embodiments, the therapy comprises a surgical therapy. For example, the therapy comprises an atrial septostomy. In another example, the therapy comprises a heart transplant and/or a lung transplant. In such instances, the subject can be administered an immunosuppressive agent to help reduce the chance of rejection.

An effective amount of the therapy can be administered to a subject (e.g., a human) in need of the treatment via any suitable route, such as intravenous administration, e.g., as a bolus or by continuous infusion over a period of time, by intranasal, intramuscular, intraperitoneal, intracerebrospinal, subcutaneous, intra-articular, intrasynovial, intrathecal, oral inhalation, or topical routes.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or virtually any other reason.

Empirical considerations such as the half-life of an agent will generally contribute to the determination of the dosage. Frequency of administration can be determined and adjusted over the course of therapy, and is generally, but not necessarily, based on treatment and/or suppression and/or amelioration and/or delay of pulmonary hypertension (PH) (e.g., pulmonary arterial hypertension (PAH), chronic thromboembolic pulmonary hypertension (CTEPH), or acute respiratory distress syndrome (ARDS)). Alternatively, sustained continuous release formulations of therapeutic agent may be appropriate. Various formulations and devices for achieving sustained release are known in the art.

As used herein, the term “treating” refers to the application or administration of a composition including one or more active agents to a subject who has pulmonary hypertension (e.g., PAH, CTEPH, or ARDS), a symptom of pulmonary hypertension, and/or a predisposition toward pulmonary hypertension, with the purpose to cure, heal, alleviate, relieve, alter, remedy, ameliorate, improve, or affect the disorder, the symptom of the pulmonary hypertension, and/or the predisposition toward pulmonary hypertension.

Alleviating pulmonary hypertension includes delaying the development or progression of the disease, and/or reducing disease severity. Alleviating the disease does not necessarily require curative results.

As used herein, “delaying” the development of a disease (e.g., PH, PAH, CTEPH, or ARDS) means to defer, hinder, slow, retard, stabilize, and/or postpone progression of the disease. This delay can be of varying lengths of time, depending on the history of the disease and/or individuals being treated. A method that “delays” or alleviates the development of a disease and/or delays the onset of the disease is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques known in the art. However, development also refers to progression that may be undetectable. For purposes of this disclosure, development or progression refers to the biological course of the symptoms. “Development” includes occurrence, recurrence, and onset. As used herein, “onset” or “occurrence of pulmonary hypertension includes initial onset and/or recurrence.

In some embodiments, the therapy is administered one or more times to the subject. In some embodiments, the therapy comprises two or more types of therapies that can be administered as part of a combination therapy for treatment of pulmonary hypertension (e.g., a combination therapy comprising an anti-hypertensive agent and an anticoagulant).

The term combination therapy, as used herein, embraces administration of these agents in a sequential manner, that is wherein each therapeutic agent is administered at a different time, as well as administration of these therapeutic agents, or at least two of the agents, in a substantially simultaneous manner.

Sequential or substantially simultaneous administration of each agent can be affected by any appropriate route including, but not limited to, intranasal routes, oral routes, intravenous routes, intramuscular routes, subcutaneous routes, and direct absorption through mucous membrane tissues. The agents can be administered by the same route or by different routes. For example, a first agent can be administered orally, and a second agent can be administered intravenously.

As used herein, the term “sequential” means, unless otherwise specified, characterized by a regular sequence or order, e.g., if a dosage regimen includes the administration of a first therapeutic agent and a second therapeutic agent, a sequential dosage regimen could include administration of the first therapeutic agent, before, simultaneously, substantially simultaneously, or after administration of the second therapeutic agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents described herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any ways as limiting their scope.

### Materials and Methods

The following materials and methods were used in the Examples set forth herein.

### Clinical Population

The study described herein complies with the Declaration of Helsinki. Plasma samples were recruited based on availability from five PAH referral centers: Brigham and Women's Hospital (Partners A), Massachusetts General Hospital (Partners B), Rhode Island Hospital/Brown University, University of Washington, and Johns Hopkins University. At each center, consecutive patients were enrolled, based on patient consent. This study was approved by the local Institutional Review Board at each participating center. Details on the method for enrolling patients, collecting clinical data, and processing samples are provided herein.

### Definitions of PAH, Other Clinical Phenotypes, and Controls

Standardized clinical definitions were used to classify all patients (Gabe et al. ESC/ERS Guidelines for the diagnosis and treatment of pulmonary hypertension: the Joint Task Force for the Diagnosis and Treatment of Pulmonary Hypertension of the European Society of Cardiology (ESC) and the European Respiratory Society (ERS): endorsed by: Association for European Pediatric and Congenital Cardiology (AEPC), International Society for Heart and Lunch Transplantation (ISHL) Eur Heart J 2016; 37:67-119), which was informed by each participating center investigator (as above) and reviewed independently by B.A.M. For PAH, the following hemodynamic criteria were used based on the prevailing definition during the study time period: mean pulmonary artery pressure (mPAP)≥25 mmHg, PVR≥3.0 Wood units, and pulmonary artery wedge pressure (PAWP)≤15 mmHg. Patients with World Health Organization (WHO) Group 2 pulmonary hypertension (PH) had mPAP≥25 mmHg, PAWP>15 mmHg and a diagnosis of heart failure with reduced left ventricular ejection fraction, heart failure with preserved left ventricular ejection fraction or other primary cardiac pathology (Maron et al. Diagnosis, treatment, and clinical management of pulmonary arterial hypertension in the contemporary era: a review. JAMA Cardiol 2016; 1:1056-65). Patients with WHO Group 3 PH had mPAP≥25 mmHg, PAWP<15 mmHg, and a ventilatory defect detected on spirometry, obstructive sleep apnea, or other established risk factor for PH due to hypoxia or sleep disordered breathing. Idiopathic PAH (iPAH), portopulmonary hypertension (Porto-PAH), human immunodeficiency virus (HIV)-PAH, and exercise-induced PH were diagnosed by appropriate serologic data and/or clinical criteria published previously (Galiè et al. ESC/ERS Guidelines for the diagnosis and treatment of pulmonary hypertension: the Joint Task Force for the Diagnosis and Treatment of Pulmonary Hypertension of the European Society of Cardiology (ESC) and the European Respiratory Society (ERS): endorsed by: Association for European Pediatric and Congenital Cardiology (AEPC), International Society for Heart and Lunch Transplantation (ISHL) Eur Heart J 2016; 37:67-119; and Oldham et al. Network analysis to risk stratify patients with exercise intolerance. Circ Res 2018; 122:864-76). Drug- and toxin-induced PAH was diagnosed mainly in the setting of methamphetamine use according to recently published guidelines (Zamanian et al. Features and outcomes of methamphetamine-associated pulmonary arterial hypertension. Am J Respir Crit Care Med 2018; 197:788-800).

### Enzyme-Linked Immunosorbent Assay (ELISA) for NEDD9 Quantification

Human plasma samples were diluted 1:3 with PBS and NEDD9 levels were analyzed using a NEDD9 enzyme linked immunosorbent assay (ELISA) kit (Aviva Systems Biology, Corp. San Diego, Calif., #OKEH02459), according to manufacturer recommendations. All plasma NEDD9 levels were measured and analyzed in the same laboratory by a single investigator (A.O.S.). The manufacturer's reported and the investigator's observed intra-assay coefficient of variance (CV) for the ELISA assay used in this study is 4.9% and 8.4%, respectively (Table 1). The manufacturer's reported interassay CV is 8.5%; this metric could not be assessed independently due to insufficient sample quantity.

TABLE 1
Intra-assay of coefficient of variance (CV) for the NEDD9 ELISA assay
measured by the investigators in the studies described herein.

| Center | CV % ± SE |
|---|---|
| Partners A | 8.5 ± 1.2 |
| Partners B | 13 ± 1.5 |
| Rhode Island Hospital (RIH)/ | 9.0 ± 2.2 |
| Brown University | |
| University of Washington | 7.0 ± 0.9 |
| Johns Hopkins University | 14 ± 1.3 |
| Combined (All centers) | 11 ± 0.8 |

### Statistical Methods

All analyses were performed using Origin 9.01 (B.A.M.) or STATA v15.1 (P.J.L.). Data are expressed as mean±SEM or median (interquartile range) for normally and non-normally distributed data, respectively. The unpaired Student t-test and one-way ANOVA test were used for comparisons between two and two or more normally distributed groups, respectively. The Mann-Whitney and Kruskal-Wallis nonparametric tests were used to compare two or more non-normally distributed groups. The Pearson r and Spearman ρ coefficients are reported for linear regression analyses performed using data that was distributed normally and non-normally, respectively. Exploratory analyses of the center-specific relationship between NEDD9 and PVR were performed to evaluate heterogeneity imposed by center in the relationship between NEDD9 and a PAH-specific marker. Linear regression was used to estimate the relationship between NEDD9 and PVR. Unadjusted analyses and models accounting for age or age, sex, and PAH sub-type were considered. The area under the curve (AUC) for the receiver operating characteristic analysis was calculated using methods reported previously (Oldham et al. Network analysis to risk stratify patients with exercise intolerance. Circ Res 2018; 122:864-76). Cox proportional hazards models were used to assess the association between plasma NEDD9 and lung transplant-free survival. Kaplan-Meier event-free curves were plotted for NEDD9 dichotomized using the median plasma level in PAH patients using lung transplant or death as the event. Unadjusted group comparisons for time-to-event outcomes were made using the log-rank test.

### Patient Enrollment and Sample Processing

Partners A Cohort:

The criteria for enrollment used at this site have been reported previously (Maron et al. Plasma aldosterone levels are elevated in patients with pulmonary arterial hypertension in the absence of left ventricular heart failure: A pilot study. Eur J Heart Fail. 2013; 15:277-83.). Patients were referred to the Pulmonary Hypertension Center between 2014-2017 for evaluation of unexplained dyspnea, unexplained exercise intolerance, or pulmonary hypertension detected by echocardiography, or to assess response to PAH-specific pharmacotherapy. All patients underwent an elective supine right heart catheterization, generally following a 6-8 hour period a nil per os. Whole blood was accessed from the pulmonary artery compartment and immediately centrifuged at 1200 r.p.m. for 10 min at 4° C. The plasma fraction was isolated and stored immediately at −80° C. For proteomic analysis of NEDD9 by mass spectrometry, whole blood was provided by a healthy volunteer (B.A.M.).

Partners B Cohort:

The criteria for enrollment used at this site have been reported previously (Malhotra et al. Circulating angiogenic modulatory factors predict survival and functional class in pulmonary arterial hypertension. Pulm Circ. 2013; 3:369-80; and Nikolic et al. Bone morphogenetic protein 9 is a mechanistic biomarker of portopulmonary hypertension. Am J Respir Crit Care Med. 2019; 199:891-902). Patients were referred to the Pulmonary Hypertension Service from 2010 to 2013 for evaluation of unexplained dyspnea, exercise intolerance, or evidence of pulmonary hypertension by echocardiography, or to assess responses to PH-specific pharmacotherapy. All patients underwent elective supine right heart catheterization. Whole blood was sampled from the right ventricular compartment into EDTA-containing tubes, immediately centrifuged at 1000 g for 20 min at 4° C., and the plasma fraction isolated and stored immediately at −80° C. until use.

Rhode Island Hospital/Brown University:

Participants were recruited from the Rhode Island Hospital Pulmonary Hypertension Center at Brown University local registry from 2012 and 2015. This biobank and database includes PH patients of all groups; however, for the current study, a subset of thoroughly characterized PAH patients was selected. Blood was obtained by peripheral venipuncture into EDTA vacutainers and centrifuged at 2500 g for 15 minutes. The plasma fraction was then removed, aliquoted and stored immediately at −80° C.

University of Washington:

Participants were recruited from the Pulmonary Vascular Disease Program into the Seattle Right Ventricle Translational Science Study (Servetus) from April of 2014 until May of 2016. At the time of enrollment all participants were determined to have PAH using standard definitions; however, <5% of the cohort on review over time were ultimately diagnosed with a non-PAH from of PH. A random subset of the Servetus participants was selected for inclusion and analysis in the current NEDD9 study. Blood was obtained by peripheral venipuncture and a total tourniquet time of <2 min. EDTA, citrate, special coagulation, and serum tubes were collected. EDTA tubes were used for the current study, were placed on the mixer for 30 s, and then stored upright on ice for no more than 30 min until being centrifuged at 2,000 g for 15 min. The plasma fraction was aliquoted into 1 mL cryovials and stored immediately at −80° C.

Johns Hopkins University:

Details on enrollment, cardiac magnetic resonance imaging (CMR), and invasive cardiopulmonary exercise testing have been reported in detail previously (Hsu et al. Right ventricular functional reserve in pulmonary arterial hypertension. Circulation 2016; 133:2413-22). Briefly, patients were referred for right heart catheterization between 2012-2017 for evaluation of dyspnea or pulmonary hypertension. All patients underwent catheterization following at least a 4-hour period of nil per os. Whole blood was accessed from the right ventricle, subsequently centrifuged at 2,000×g for 10 min at 4° C., and the plasma fraction was isolated and stored immediately at −80° C.

### Outcome Data

Time-to-events were calculated as the interval between blood draw for NEDD9 analysis and either all-cause mortality or lung-transplantation for patients experiencing an end-point, or last clinical contact for patients censured from the analysis. These data were produced by a co-author from each participating center, who used direct patient contact at regular intervals, the electronic health record, the social security death index, or other standard means by which to track events.

### Patient Subpopulation Criteria

In studies described herein, the CTD-PAH patients had serologic evidence of systemic sclerosis, mixed connective tissue disease, systemic lupus erythematosus, or rheumatoid arthritis and/or were diagnosed by a board-certified rheumatologist. In selected cases, a single primary diagnosis was unclear and patients were classified as a mixed phenotype. These patients were grouped with WHO Group 5 PH patients in this study. Patients were classified as dyspnea non-PH controls if their untreated cardiopulmonary hemodynamic profile was mPAP<25 mmHg, PVR<3.0 Wood units, and PAWP<15 mmHg, and exercise-induced PH was not diagnosed previously. Patients with chronic thromboembolic disease or chronic thromboembolic pulmonary hypertension were excluded from this study based mainly on low availability.

### Cardiac MRI (CMR)

CMR with delayed gadolinium contrast enhancement was performed on a 3-T MRI system (Magnetom Trio, Siemens Healthcare, Erlangen, Germany) within 6 hours prior to right heart catheterization.

### Invasive Cardiopulmonary Exercise Testing

After completion of a baseline right heart catheterization, a 4F pulmonary artery catheter was placed in the pulmonary artery position. Subjects were then positioned into a supine bicycle ergometer. A nose clip and mouthpiece (Innocor, Innovision, Denmark) was used to record continuous metabolic gas exchange. Subjects then underwent staged bicycle exercise, beginning at 15 W in stage 1, and increasing by 10 W increments per 2-min stage until a symptom-limited maximum was achieved. Hemodynamic pressure, gas exchange, blood oximetry, and direct Fick cardiac output are reported in this study at peak exercise.

### Laboratory Tests

Blood creatinine, hemoglobin, and N-terminal brain natriuretic peptide (NT-BNP) values were analyzed at a CLIA-certified pathology laboratory located at the respective participating centers.

### Plasma NEDD9 Measurement

Immunoblot:

Proteins were size-fractionated electrophoretically by sodium dodecyl sulfate polyacrylamide gel (SDS-PAGE) using the Mini-protean TGX™ Gel system (Bio-Rad, #456-1084), and transferred to polyvinylidene fluoride membranes according to methods reported previously (Maron et al. Aldosterone inactivates the endothelin-B receptor to decrease pulmonary endothelial nitric oxide levels and modulate pulmonary arterial hypertension. Circulation 2012; 126(8):963-974). The membranes were then incubated with an anti-NEDD9 antibody (Abcam, catalogue #ab18056; source is mouse; recognizes mouse, rat, and human NEDD9 [dilution 1:1000]) (Samokhin et al. NEDD9 targets COL3A1 to promote endothelial fibrosis and pulmonary arterial hypertension. Sci Transl Med. 2018; 10:445). The immunogen for this antibody corresponds to amino acids 82-398 of human NEDD9 (Samokhin et al. NEDD9 targets COL3A1 to promote endothelial fibrosis and pulmonary arterial hypertension. Sci Transl Med. 2018; 10:445).

Liquid Chromatography-Mass Spectrometry (LC-MS):

For LC-MS analysis, 10 μL of human plasma were immunoprecipitated using a mouse monoclonal NEDD9 antibody (Abcam, #ab18056) and SureBeads Protein G Magnetic Beads (Bio-Rad, #161-4023), according to manufacturer recommendations. Proteins were size-fractionated electrophoretically (by SDS-PAGE and immunoprecipitates were stained using Bio-Safe™ Coomassie G-250 Stain (Bio-Rad, #161-0786). The 105-115 kDa band was cut into approximately 1 mm³ pieces, stored in ice cold PBS, and analyzed by LC-MS within 7 days.

Gel pieces were then subjected to a modified in-gel trypsin digestion procedure: the gel pieces were washed and dehydrated with acetonitrile for 10 min followed by removal of acetonitrile (Shevchenko et al. Mass spectrometric sequencing of proteins from silver-stained polyacrylamide gels. Anal Chem. 1996; 68:850-858; and Maron et al. Aldosterone increases oxidant stress to impair guanylyl cyclase activity by cysteinyl thiol oxidation in vascular smooth muscle cells. J Biol Chem. 2009; 284:7665-7672). Pieces were then completely dried in a speed-vac. Rehydration of the gel pieces was with 50 mM ammonium bicarbonate solution containing 12.5 ng/μl modified sequencing-grade trypsin (Promega, Madison, Wis.) at 4° C. After 45 min, the excess trypsin solution was removed and replaced with 50 mM ammonium bicarbonate solution to just cover the gel pieces. Samples were then placed in a 37° C. room overnight. Peptides were later extracted by removing the ammonium bicarbonate solution, followed by one wash with a solution containing 50% acetonitrile and 1% formic acid. The extracts were then dried in a speed-vac (˜1 hr) and stored at 4° C. until analysis.

On the day of analysis, the samples were reduced with DTT (Sigma) at a 1 mM concentration (in 50 mM ammonium bicarbonate) for 30 min at 60° C. The samples were then cooled to room temperature and iodoacetamide (stock in 50 mM ammonium bicarbonate) (Sigma) was added to a concentration of 5 mM for 15 min in the dark at room temperature. DTT was then added to a 5 mM concentration to quench the reaction. Sequence grade trypsin was then added at a concentration of 5 ng/μ1. The digestion step was over-night at 37° C. The samples were then desalted by a custom-made desalting column. Samples were reconstituted in 5-10 μl of HPLC solvent A (97.5% water, 2.5% acetonitrile and 0.1% formic acid). A nano-scale reverse-phase HPLC capillary column was created by packing 2.6 μm C18 spherical silica beads into a fused silica capillary (100 μm inner diameter×—30 cm length) with a flame-drawn tip (Peng et al. Proteomics: The move to mixtures. J Mass Spec. 2001; 36:1083-91.). After equilibrating the column each sample was loaded via a Famos auto sampler (LC Packings, San Francisco Calif.) onto the column. A gradient was formed and peptides were eluted with increasing concentrations of solvent B (HPLC buffer B=97.5% acetonitrile, 2.5% water and 0.1% formic acid).

As peptides eluted, they were subjected to electrospray ionization and then entered into an LTQ Orbitrap Velos Pro ion-trap mass spectrometer (Thermo Fisher Scientific, Waltham, Mass.). Peptides were detected, isolated, and fragmented to produce a tandem mass spectrum of specific fragment ions for each peptide. Peptide sequences (and hence protein identity) were determined by matching protein databases with the acquired fragmentation pattern by the software program, Sequest (Thermo Fisher Scientific, Waltham, Mass.) (Eng et al. An approach to correlate tandem mass spectral data of peptides with amino acid sequences in a protein database. J Am Soc Mass Spectrom. 1994; 5:976-989). All databases include a reversed version of all the sequences and the data was filtered to between a one and two percent peptide false discovery rate.

### Cell Culture

Male human pulmonary artery endothelial cells (Lonza) were cultured in medium supplemented with 5% fetal bovine serum and incubated at 37° C., 5.0% CO₂ and passaged twice-weekly using 0.5% trypsin/EDTA as reported previously (Samokhin et al. NEDD9 targets COL3A1 to promote endothelial fibrosis and pulmonary arterial hypertension. Sci Transl Med. 2018; 10:445). Only passage 3-8 cells were used for experiments.

### Anti-NEDD9 Immunoprecipitation

Immunoprecipitation was performed on HPAEC monolayers using an anti-NEDD9 antibody (Abcam, catalogue #ab18056) according to methods reported previously by our laboratory (Maron et al. Aldosterone inactivates the endothelin-B receptor to decrease pulmonary endothelial nitric oxide levels and modulate pulmonary arterial hypertension. Circulation 2012; 126(8):963-974; and Samokhin et al. NEDD9 targets COL3A1 to promote endothelial fibrosis and pulmonary arterial hypertension. Sci Transl Med. 2018; 10:445). Serial dilution of samples prepared for ELISA was accomplished using PBS.

### Example 1: Verifying Plasma NEDD9

The ELISA assay in the current study was used previously to quantitate NEDD9 in plasma samples from patients with other diseases. However, verification of commercially purchased laboratory reagents is identified increasingly as a critical step toward optimizing scientific rigor (Wertheim et al. Isolating pulmonary microvascular endothelial cells ex vivo: implications for pulmonary arterial hypertension, and a caution on the use of commercial biomaterials. PLOS ONE 2019; 14:e0211909), and prior reports used only a single method to analyze plasma NEDD9 (Karabulut et al. Serum neural precursor cell-expressed, developmentally down regulated 9 (NEDD9) level may have a prognostic role in patients with gastric cancer. Biomed Pharmacother 2015; 73:140-6).

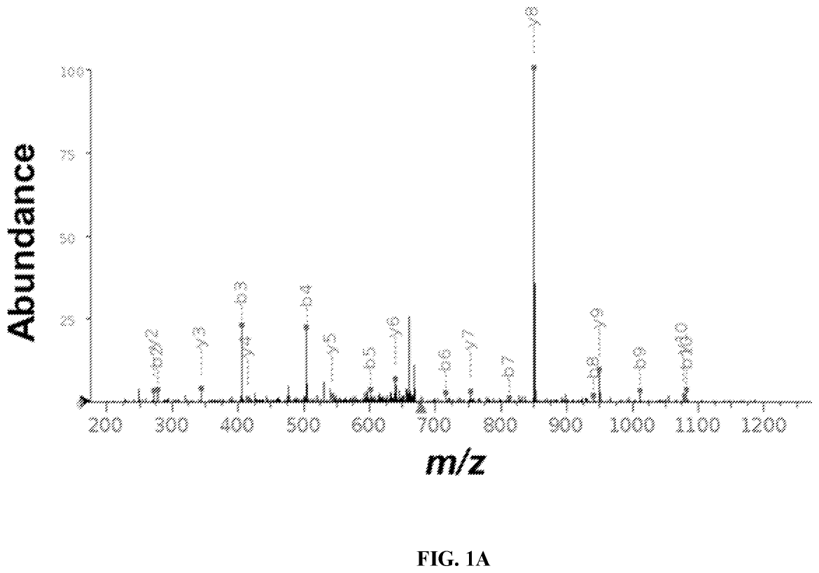

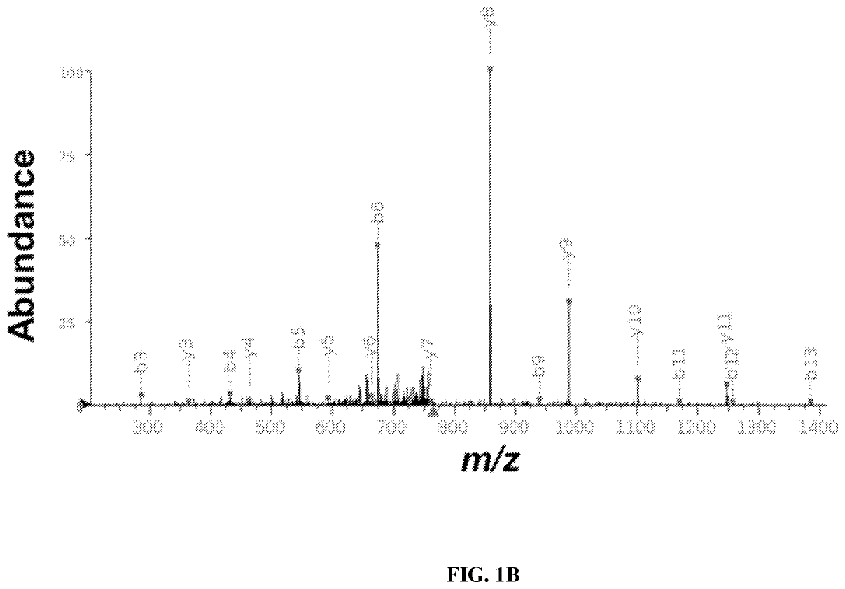

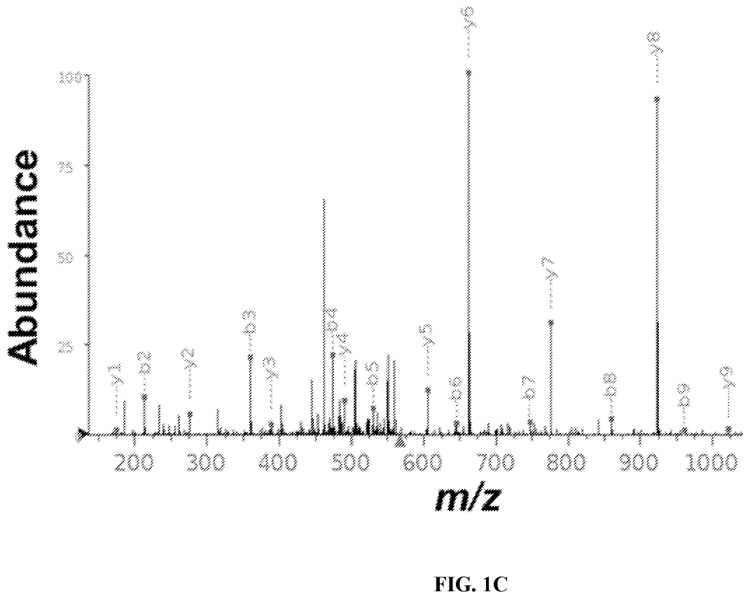

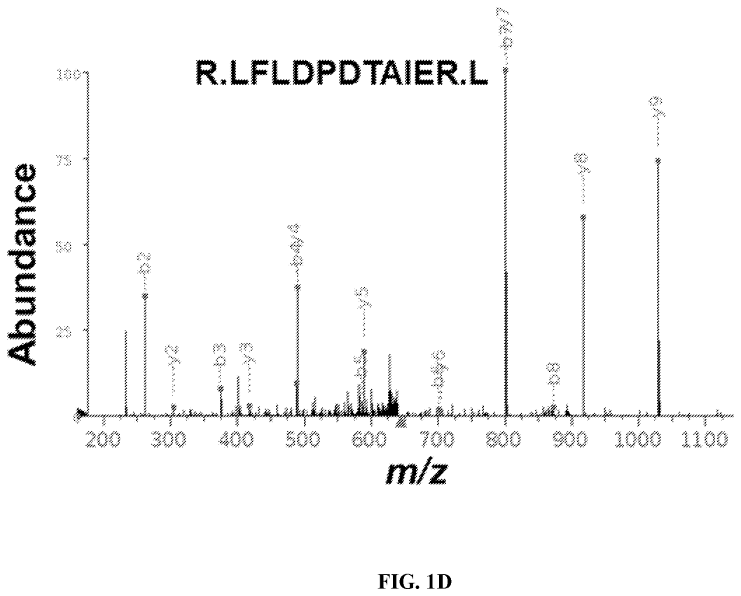

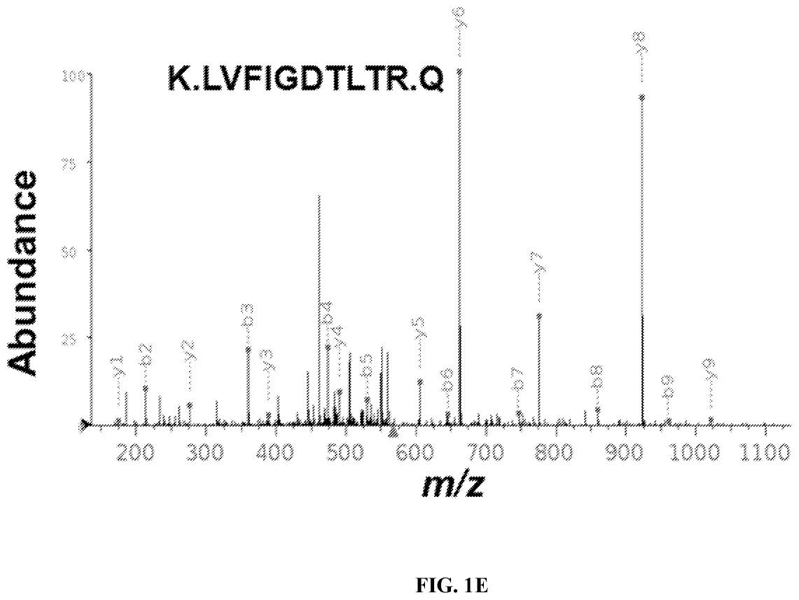

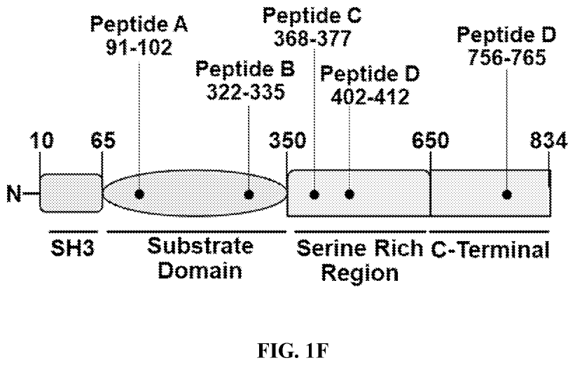

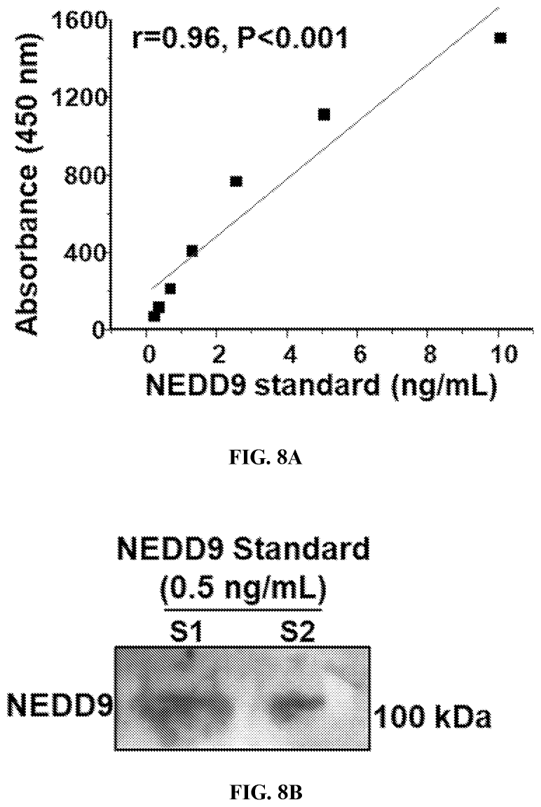

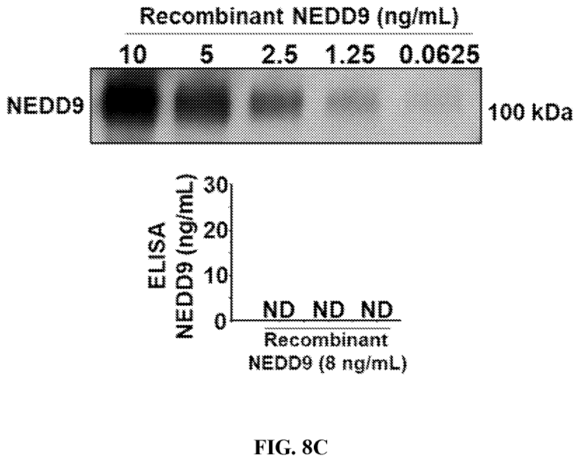

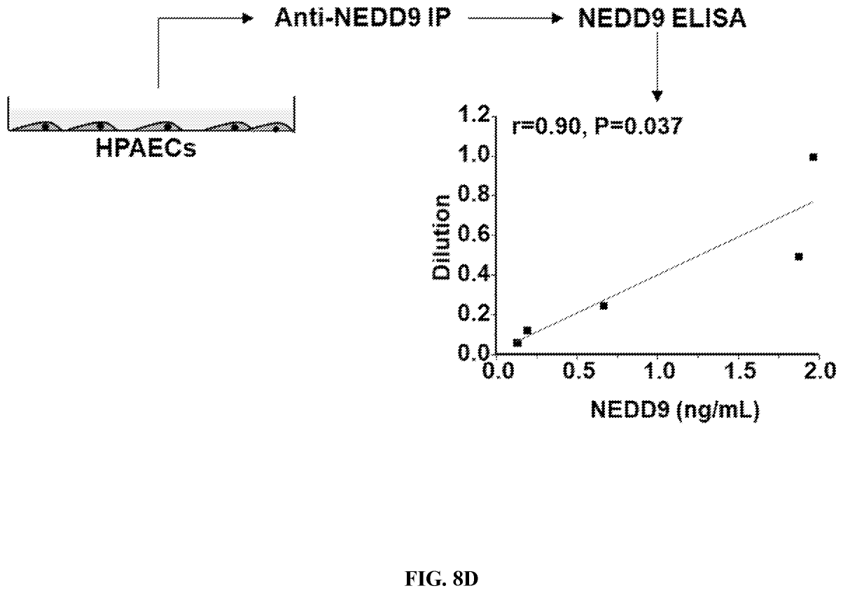

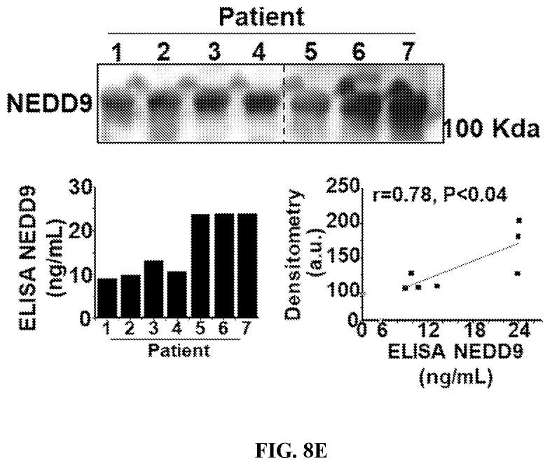

Studies described herein confirmed detection of NEDD9 in plasma using LC-MS, which is the gold-standard method for proteomic analysis. This approach detected 5 peptides within 3 of the 4 distinct NEDD9 domains (N≥2 replicates of each peptide per sample; N=2 samples) (FIGS. 1A-1F). This was then confirmed further by anti-NEDD9 immunoblot using an antibody targeting the peptide sequence spanning amino acids 85-350 (FIGS. 8A-8E), which was a region of the protein detected by LC-MS (N=3).

### Example 2: NEDD9 Plasma Sample Throughput

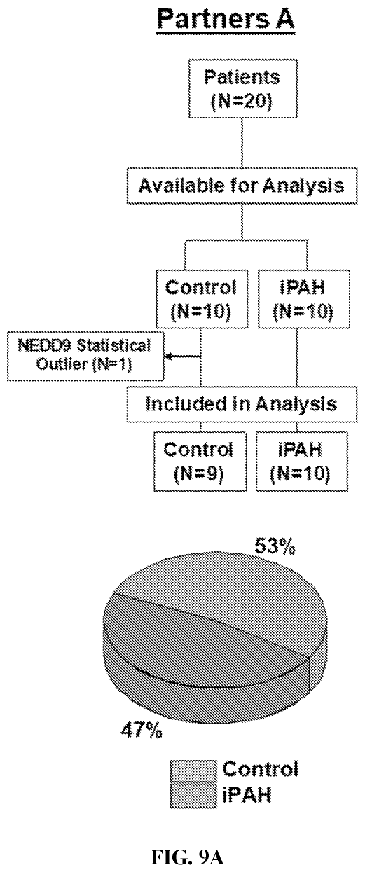

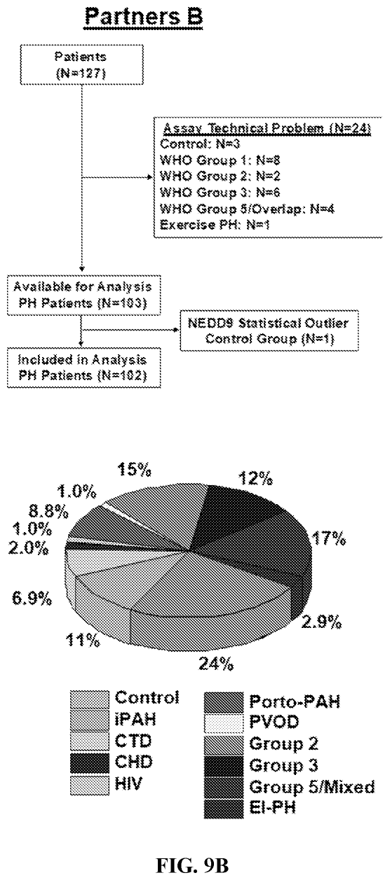

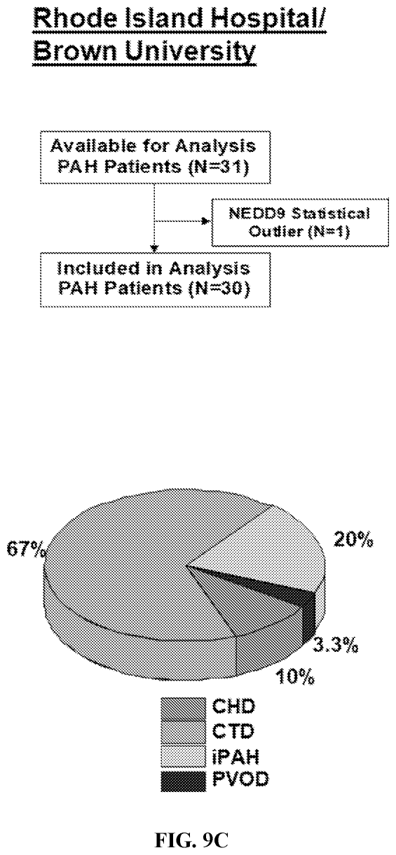

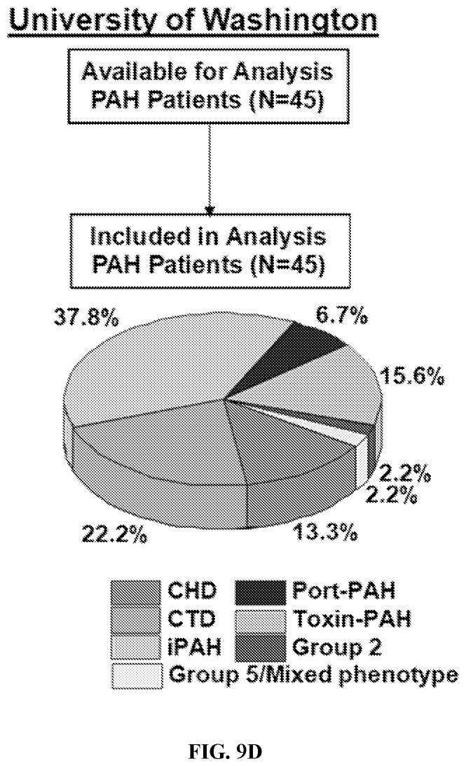

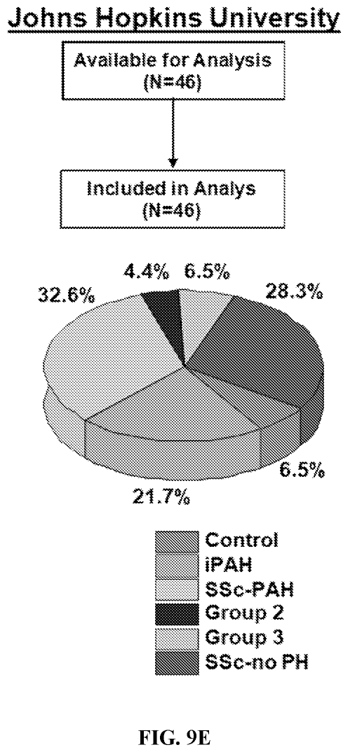

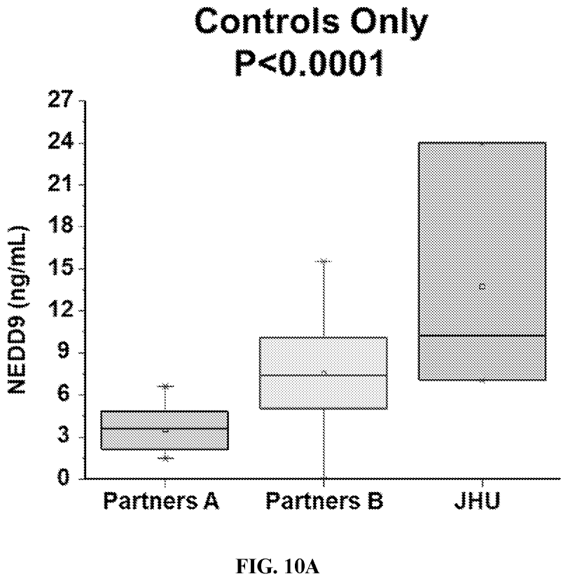

From a total of 265 individual patient plasma samples, 239 (90.2%) were available for analyzing the clinical relevance of NEDD9 after excluding one assay (inclusive only of Partners B samples) that was technically unsound (defined by biologically implausible values) (N=23) and statistical outliers (N=3) (Table 2 and FIGS. 9A-9E).

TABLE 2
Summary of patient diagnosis for the study cohort. Partners A is Brigham and Women’s
Hospital; Partners B is Massachusetts General Hospital; PAH, pulmonary arterial
hypertension of any subtype; WHO, World Health Organization; SSc, systemic
sclerosis; EI-PH, exercise-induced pulmonary hypertension.

| | | | | | WHO | | | |
|---|---|---|---|---|---|---|---|---|
| | | | | | Group 5/ | | | |
| | | | WHO | WHO | Mixed | SSc-no | EI- | |
| Center | Control | PAH | Group 2 | Group 3 | Phenotype | PH | PH | Total |
| Partners A | 9 | 10 | 0 | 0 | 0 | 0 | 0 | 19 |
| Partners B | 24 | 31 | 15 | 12 | 17 | 0 | 3 | 102 |
| Warren Alpert/ | 0 | 30 | 0 | 0 | 0 | 0 | 0 | 30 |
| Brown University | | | | | | | | |
| University of | 0 | 43 | 1 | 0 | 1 | 0 | 0 | 45 |
| Washington | | | | | | | | |
| Johns Hopkins | 3 | 25 | 2 | 3 | 0 | 13 | 0 | 46 |
| University | | | | | | | | |
| Total | 36 | 139 | 18 | 15 | 18 | 13 | 3 | 239 |

### Example 3: Study Population

Compared to controls, PAH patients (82% female) had higher mPAP (17 [13-19] vs. 45 [35-57] mmHg, P<0.0001), PVR (94.6 [68.0-137] vs. 385.8 [229.1-717.3] dynes*s*cm⁵, P<0.0001) and transpulmonary gradient (7 [4-9] vs. 35 [24-48] mmHg, P<0.0001) as expected, but were not significantly different in age (52.9 [41.5-66.8] vs. 59.0 [46.1-67.3] yr, P=0.31) (Table 3 and Table 5). Clinical comorbidities for each cohort are available in Tables 6-10. The pharmacotherapeutic profile was available for 92.8% of PAH patients (N=129), from which PAH-specific medication was reported in 42.6% (N=55), indicating a mixture of prevalent and incident patients in the study cohort. Phosphodiesterase type-V inhibitors, endothelin receptor antagonists, and prostacyclin replacement drugs were recorded for 86%, 53%, and 48% of PAH patients, respectively (Table 4 and Tables 11-15).

TABLE 3
Hemodynamic characteristics of the initial control and pulmonary arterial hypertension (PAH)
cohorts. Data were collected on RAP, right atrial pressure; mPAP, mean pulmonary artery
pressure; TPG, transpulmonary gradient; PVR, pulmonary vascular resistance; PAWP,
pulmonary artery wedge pressure; CO, cardiac output, CI, cardiac index; PH, pulmonary
hypertension. Data are expressed as median (IQR). P-Value data are from ANOVA analysis.

| | | | | Clinical and Hemodynamic | | | |
|---|---|---|---|---|---|---|---|
| | Patients (N) | | | Variables by Group | | | |
| | | | Non-PAH | | | Non-PAH | |
| Variable | Control | PAH | PH | Control | PAH | PH | P-Value |
| Age | 37 | 142 | 54 | 52.9 | 59.0 | 63.0 | 0.0027 |
| | | | | (41.5-66.8) | (46.1-67.3) | (54.4-71.1) | |
| RAP (mmHg) | 33 | 126 | 52 | 3 | 8 | 9 | <0.0001 |
| | | | | (2-6) | (5-12) | (5.5-14) | |
| mPAP (mmHg) | 35 | 134 | 54 | 17 | 45 | 37.5 | <0.0001 |
| | | | | (13-19) | (35-57) | (30-46) | |
| TPG (mmHg) | 36 | 134 | 54 | 7 | 35 | 20 | <0.0001 |
| | | | | (4-9) | (24-48) | (15-25) | |
| PVR | 35 | 118 | 53 | 94.6 | 624.0 | 295.9 | <0.0001 |
| (dynes*s*cm⁵) | | | | (68.0-137) | (350.0-808.0) | (217.0-389.0) | |
| PAWP | 28 | 125 | 54 | 10 | 10 | 18 | <0.0001 |
| (mmHg) | | | | (6.0-12) | (8-13) | (11-25) | |
| CO (L/min) | 35 | 114 | 53 | 5.3 | 4.6 | 5.0 | 0.1924 |
| | | | | (4.3-6.7) | (3.8-5.9) | (4.0-6.3) | |
| CI (L/min/m²) | 32 | 39 | 47 | 2.8 | 2.5 | 2.7 | 0.0511 |
| | | | | (2.5-3.6) | (2.1-3.0) | (2.2-3.2) | |

TABLE 4
PAH medical regimen for the overall pulmonary
arterial hypertension (PAH) cohort.
Total PAH Cohort (N = 139)

| Medication | | |
|---|---|---|
| Calcium channel antagonist | 20 | (14) |
| PDE-Vi | 86 | (61) |
| ERA | 53 | (37) |
| Prostacyclin therapy | 48 | (34) |
| sGC therapy | 0 | (0) |
| Supplemental O₂ | 16 | (11) |
| ACE-inhibitor/ARB | 14 | (10) |
| Anticoagulant | 29 | (21) |
| Diuretic | 82 | (59) |
| MRA | 24 | (17) |

PDE, phosphodiesterase; ERA, endothelin receptor antagonist; sGC, soluble guanylyl cyclase; ACE, angiotensin converting enzyme; ARB, angiotensin receptor blocker; MRA, mineralocorticoid receptor antagonist. Data are presented as N (%).

TABLE 5
Patient characteristics of the PAH and control study population. The frequency
(N) (top) and quantification (bottom) of data for selected demographic, clinical, and
hemodynamic characteristics of patients with pulmonary arterial hypertension (PAH) of any
subtype and non-disease controls are presented by participating center. (Bottom) The
distribution of pulmonary hypertension subgroup prevalence by center is provided in FIGs.
9A-9E. PAH, pulmonary arterial hypertension; RIH, Rhode Island Hospital; UW, University
of Washington; PHA, Pulmonary Hypertension Association; BMI, body mass index; HR,
heart rate; RAP, right atrial pressure; mPAP, mean pulmonary artery pressure; PAWP,
pulmonary artery wedge pressure; MvO₂ mixed venous oxygen saturation; CO, cardiac
output; CI, cardiac index; PVR, pulmonary vascular resistance. PA, pulmonary artery; PV,
peripheral vein. In the bottom table, data are presented as mean (SD) and median [IQR]
normally and non-normally distributed data, respectively.

| | | | | | | | | | | | |
|---|---|---|---|---|---|---|---|---|---|---|---|
| | Total | | | | | | | | | | |
| Cohort | N | Age | Sex | BMI | RAP | mPAP | PAWP | MvO₂ | CO | CI | PVR |

Partners A

| | | | | | | | | | | | |
|---|---|---|---|---|---|---|---|---|---|---|---|
| Control | 9 | 9 | 9 | 9 | 9 | 9 | 9 | 9 | 9 | 9 | 9 |
| PAH | 10 | 10 | 10 | 10 | 10 | 10 | 10 | 10 | 10 | 10 | 10 |

Partners B

| | | | | | | | | | | |
|---|---|---|---|---|---|---|---|---|---|---|
| Control | 24 | 24 | 24 | 16 | 20 | 23 | 24 | 23 | 23 | 22 |
| PAH | 31 | 31 | 31 | 27 | 29 | 31 | 31 | 30 | 30 | 30 |

RIH/Brown University

| | | | | | | | | | | |
|---|---|---|---|---|---|---|---|---|---|---|
| PAH | 30 | 30 | 30 | 30 | 30 | 30 | 30 | 29 | 28 | 28 |

UW

| | | | | | | | | | | | |
|---|---|---|---|---|---|---|---|---|---|---|---|
| PAH | 43 | 43 | 43 | 42 | 30 | 35 | 28 | 26 | 21 | 21 | 23 |

JHU

| | | | | | | | | | | | |
|---|---|---|---|---|---|---|---|---|---|---|---|
| Control | 3 | 3 | 3 | 3 | 3 | 3 | 3 | 3 | 3 | 3 | 3 |
| PAH | 25 | 25 | 25 | 25 | 25 | 25 | 25 | 25 | 25 | 25 | 25 |

TABLE 6
Comorbidities for BWH cohort.
Partners A Cohort

| Control | Comorbidity | iPAH | Comorbidity |
|---|---|---|---|
| Patient #1 | Autonomic neuropathy | Patient #1 | Atrial fibrillation |
| | | | Hypothyroidism |
| | | | Prior pulmonary embolism |
| | | | Obstructive sleep apnea |
| Patient #2 | pSVT | Patient #2 | IBS |
| | GERD | | Osteoporosis |
| Patient #3 | Thyroiditis | Patient #3 | Systemic Hypertension |
| | Lyme disease | | GERD |
| | Obstructive sleep apnea | | Hyperlipidemia |
| | Asthma | | |
| | Systemic hypertension | | |
| Patient #4 | AF, Benign paroxysmal | Patient #4 | Deep vein thrombosis |
| | positional vertigo | | |
| Patient #5 | Autonomic dysfunction | Patient #5 | Diverticulitis |
| | | | Gout |
| | | | Anemia |
| | | | Systemic hypertension |
| Patient #6 | POTS | Patient #6 | Systemic hypertension |
| | | | GERD |
| Patient #7 | Systemic hypertension | Patient #7 | Deep vein thrombosis |
| | Psoriasis | | Dyslipidemia |
| | | | GERD |
| | | | Prostate cancer |
| Patient #8 | POTS | Patient #8 | Obesity, depression |
| Patient #9 | Chronic fatigue syndrome | Patient #9 | Diabetes mellitus |
| | | | Systemic hypertension |
| | | | GERD |
| | | | Hypercholesterolemia |
| | | | Anemia |
| Patient #10 | Diabetes mellitus | Patient #10 | Hyperlipidemia |
| | Autonomic dysfunction | | Systemic hypertension |
| | Systemic hypertension | | Obstructive sleep apnea |
| | Asthma | | |
| | Obstructive sleep apnea | | |

pSVT, paroxysmal supraventricular tachycardia; GERD, gastroesophageal reflux disease; POTS, postural orthostatic tachycardia syndrome; IBS, irritable bowel syndrome.

TABLE 7
Comorbidities of PAH and control patients in the Partners B cohort.
Partners B Cohort

| Control | Comorbidity | iPAH | Comorbidity |
|---|---|---|---|
| Patient #1 | AF, Hypothyroid | Patient #1 | — |
| Patient #2 | Systemic sclerosis | Patient #2 | Lymphoma |
| Patient #3 | Rheumatoid arthritis | Patient #3 | Atrial fibrillation |
| | Raynaud's | | |
| Patient #4 | Asthma | Patient #4 | |
| Patient #5 | Systemic lupus erythematosus | Patient #5 | Hodgkin lymphoma |
| Patient #6 | OSA | Patient #6 | — |
| | Interstitial lung disease | | |
| Patient #7 | COPD | Patient #7 | AF |
| | | | Nonischemic cardiomyopathy |
| | | | (viral) COPD |
| Patient #8 | Interstitial lung disease | Patient #8 | Hyperthyroidism |
| | Hemangioma | | |
| | Prior bilateral lung transplant | | |
| Patient #9 | Pulmonary sarcoidosis | Patient #9 | Uterine cancer |
| Patient #10 | Cystic fibrosis | Patient #10 | Schizophrenia |
| | Prior bilateral lung transplant | | |
| Patient #11 | Pulmonary embolism | Patient #11 | Coronary artery disease |
| | | | Diabetes mellitus type II |
| Patient #12 | Alcoholic cirrhosis | Patient #12 | — |
| | Hepatocellular carcinoma | | |
| | Prior liver transplant | | |
| Patient #13 | COPD | Patient #13 | — |
| | DVT | | |
| Patient #14 | Hepatic cirrhosis | Patient #14 | — |
| | Hemangioma | | |
| | Hepatitis C virus | | |
| Patient #15 | Autoimmune hepatitis | Patient #15 | Diabetes mellitus Type II |
| Patient #16 | Systemic sclerosis | Patient #16 | Anemia |
| | Hypothyroidism | | |
| | Raynaud's | | |
| Patient #17 | ESRD | Patient #17 | — |
| | Diabetes mellitus type II | | |
| | Hypertrophic obstructive | | |
| | cardiomyopathy | | |
| | OSA | | |
| Patient #18 | Hepatic cirrhosis | Patient #18 | Luminal PE |
| | Hepatocellular carcinoma | | Factor V Leiden heterozygous |
| | Diabetes mellitus type II | | |
| Patient #19 | Idiopathic pulmonary fibrosis | Patient #19 | — |
| | Nonspecific interstitial pneumonia | | |
| | Prior bilateral lung transplant | | |
| Patient #20 | COPD | Patient #20 | Mild obstructive lung disease |
| | Alcoholic cirrhosis | | |
| | Hemochromatosis | | |
| Patient #21 | Hashimoto's thyroiditis | Patient #21 | — |
| Patient #22 | Idiopathic pulmonary fibrosis | Patient #22 | ETOH |
| | HIV | | |
| | Fatty liver disease | | |
| Patient #23 | Hantavirus pulmonary syndrome | Patient #23 | HCV |
| | Hepatitis C virus | | |
| | Alcohol abuse | | |
| Patient #24 | Idiopathic pulmonary fibrosis | Patient #24 | HCC |
| | Coronary artery disease | | |
| | Single left lung transplant | | |
| Patient #25 | Connective tissue disease | Patient #25 | HCV, ETOH, |
| | | | thrombocytopenia |
| | | Patient #26 | HCV, thrombocytopenia |
| | | Patient #27 | Upper GI bleed |
| | | Patient #28 | HCV, thrombocytopenia |
| | | Patient #29 | Mild OSA, thrombocytopenia |
| | | Patient #30 | HCC, thrombocytopenia |
| | | Patient #31 | PCH |

AF, atrial fibrillation; OSA, obstructive sleep apnea, COPD, chronic obstructive pulmonary disease; ESRD, end-stage renal disease; HIV, human immunodeficiency virus; ETOH, alcohol misuse/abuse; HCC, hepatocellular carcinoma; HCV; hepatitis C virus; PCH, pulmonary capillary hemangiomatosis; PE, pulmonary embolism; GI, gastrointestinal; DVT, deep vein thrombosis.

TABLE 8
Comorbidities of PAH patients in the University of Washington.
University of Washington Cohort (N = 49)

| Comorbidity | N (%) | |
|---|---|---|
| Emphysema | 3 | (6) |
| Interstitial lung disease | 4 | (8) |
| Scleroderma | 7 | (14) |
| SLE | 0 | (0) |
| Rheumatoid arthritis | 3 | (6) |
| Sjogren's syndrome | 3 | (6) |
| Dysthyroid | 10 | (20) |
| Sickle cell disease | 0 | (0) |
| Asthma | 5 | (10) |
| Arthritis (NOS) | 14 | (29) |
| Rheumatic heart disease | 2 | (4) |
| Cancer | 6 | (12) |
| Primary liver disease | 5 | (10) |
| Hepatic cirrhosis | 4 | (8) |
| Chronic kidney disease | 6 | (12) |
| Hemodialysis | 0 | (0) |
| Systemic hypertension | 19 | (39) |
| Hypercholesterolemia | 12 | (25) |
| Diabetes mellitus | 5 | (10) |

SLE, systemic lupus erythematous; NOS, not otherwise specified.

TABLE 9
Comorbidities of patients in the Johns Hopkins University cohort. No comorbidities
were reported for patients with a diagnosis of systemic sclerosis and interstitial lung
disease (SSc-ILD) (N = 3). For SSc no PH and iPAH, data are presented as N (%).
HFpEF, heart failure with preserved ejection fraction; HTN, hypertension; VTE,
Venothromboembolic disease; COPD, chronic obstructive pulmonary disease; OSA,
obstructive sleep apnea. iPAH, idiopathic pulmonary arterial hypertension; PH,
pulmonary hypertension.
Johns Hopkins University Cohort (N = 25)

| | | SSc (no PH) | | | | | |
|---|---|---|---|---|---|---|---|
| Control (N = 3) | | (N = 13) | | iPAH (N = 9) | | SSc-LHD | |
| Patient | Systemic HTN | VTE | 1 (7.7) | OSA | 1 | Patient #1 | Systemic |
| #1 | HFpEF | | | | (11) | | HTN |
| | | | | | | | HFpEF |
| | | | | | | | COPD |
| Patient | — | Systemic | 2 (15) | Systemic | 1 | Patient #2 | HFpEF |
| #2 | | HTN | | HTN | (11) | | |
| Patient | — | Asthma | 1 (7.7) | | | | |
| #3 | | COPD | 1 (7.7) | | | | |

TABLE 10
Comorbidities of PAH patients in the University of Washington.
RIH/Brown University (N = 31)

| Comorbidity | N (%) | |
|---|---|---|
| Emphysema | 1 | (3.2) |
| Interstitial lung disease | 6 | (19) |
| COPD | 5 | (16) |
| Pulmonary embolism | 1 | (3) |
| Asthma | 2 | (6) |
| SLE | 1 | (3) |
| Myocardial infarction | 1 | (3) |
| Valvular heart disease | 2 | (6) |
| Systemic hypertension | 9 | (29) |
| Diabetes mellitus | 5 | (16) |
| Cancer | 5 | (16) |
| Hepatic cirrhosis | 1 | (3) |
| Chronic kidney disease | 1 | (3) |
| Thyroid disease | 6 | (19) |

SLE, systemic lupus erythematous; COPD, chronic obstructive pulmonary disease. RIH, Rhode Island Hospital.

TABLE 11
PAH medical regimen for the Partners A Cohort.
Partners A Cohort

| Medication | | |
|---|---|---|
| Calcium channel antagonist | 1 | (5) |
| PDE-Vi | 5 | (25) |
| ERA | 4 | (20) |
| Prostacyclin therapy | 3 | (15) |
| sGC therapy | 0 | (0) |
| Supplemental O₂ | 0 | (0) |
| ACE-inhibitor/ARB | 7 | (35) |
| Anticoagulant | 2 | (10) |
| Diuretic | 6 | (30) |
| MRA | 3 | (15) |

Pulmonary arterial hypertension; WHO, World Health Organization; PDE, phosphodiesterase; ERA, endothelin receptor antagonist; sGC, soluble guanylyl cyclase; ACE, angiotensin converting enzyme; ARB, angiotensin receptor blocker; MRA, mineralocorticoid receptor antagonist. Data are presented as N (%).

TABLE 12
PAH medical regimen for the Partners B Cohort.
Partners B Cohort

| Medication | | |
|---|---|---|
| Calcium channel antagonist | 3 | (10) |
| PDE-Vi | 13 | (42) |
| ERA | 6 | (19) |
| Prostacyclin therapy | 10 | (32) |
| sGC therapy | 0 | (0) |
| Supplemental O₂ | 6 | (19) |
| ACE-inhibitor/ARB | 2 | (6) |
| Anticoagulant | 6 | (19) |
| Diuretic | 9 | (29) |

Pulmonary arterial hypertension; WHO, World Health Organization; PDE, phosphodiesterase; ERA, endothelin receptor antagonist; sGC, soluble guanylyl cyclase; ACE, angiotensin converting enzyme; ARB, angiotensin receptor blocker; MRA, mineralocorticoid receptor antagonist. Data are presented as N (%).

TABLE 13
PAH medical regimen for the University of Washington Cohort.
University of Washington Cohort

| Medication | | |
|---|---|---|
| Calcium channel antagonist | 5 | (11) |
| PDE-Vi | 37 | (82) |
| ERA | 23 | (52) |
| Prostacyclin therapy | 23 | (52) |
| sGC therapy | 0 | (0) |
| Supplemental O₂ | 10 | (22) |
| ACE-inhibitor/ARB | 5 | (11) |
| Anticoagulant | 12 | (27) |
| Diuretic | 29 | (65) |
| MRA | 12 | (27) |

Pulmonary arterial hypertension; WHO, World Health Organization; PDE, phosphodiesterase; ERA, endothelin receptor antagonist; sGC, soluble guanylyl cyclase; ACE, angiotensin converting enzyme; ARB, angiotensin receptor blocker; MRA, mineralocorticoid receptor antagonist. Data are presented as N (%).

TABLE 14
PAH medical regimen for the Johns Hopkins University Cohort.
Johns Hopkins University

| Medication | | |
|---|---|---|
| Calcium channel antagonist | 11 | (39) |
| PDE-Vi | 9 | (32) |
| ERA | 5 | (18) |
| Prostacyclin therapy | 0 | (0) |
| sGC therapy | 0 | (0) |
| Diuretic | 17 | (61) |
| MRA | 5 | (18) |

PDE, phosphodiesterase; ERA, endothelin receptor antagonist; sGC, soluble guanylyl cyclase; MRA, mineralocorticoid receptor antagonist. Data are presented as N (%).

TABLE 15
PAH medical regimen for the Warren Alpert (Brown University) Cohort.
Rhode Island Hospital/Brown University

| Medication | | |
|---|---|---|
| PDE-Vi | 22 | (71) |
| ERA | 15 | (48) |
| Prostacyclin therapy | 12 | (39) |
| Diuretic | 21 | (68) |
| ACE-I/ARB | 9 | (43) |
| MRA | 4 | (13) |
| Anticoagulant | 9 | (29) |

PDE, phosphodiesterase; ERA, endothelin receptor antagonist. Data are presented as N (%).

### Example 4: NEDD9 is Increased in PAH and Associates with Cardiopulmonary Hemodynamic Severity

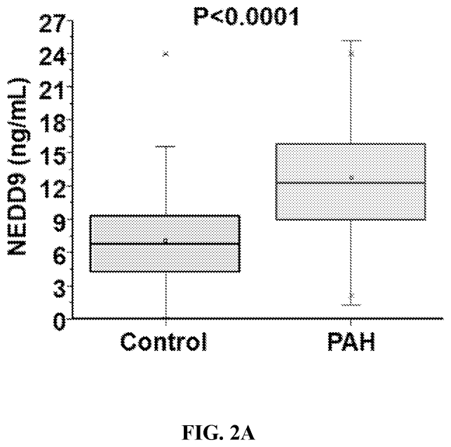

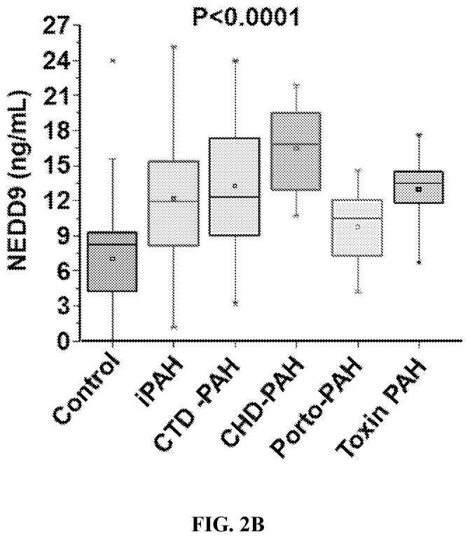

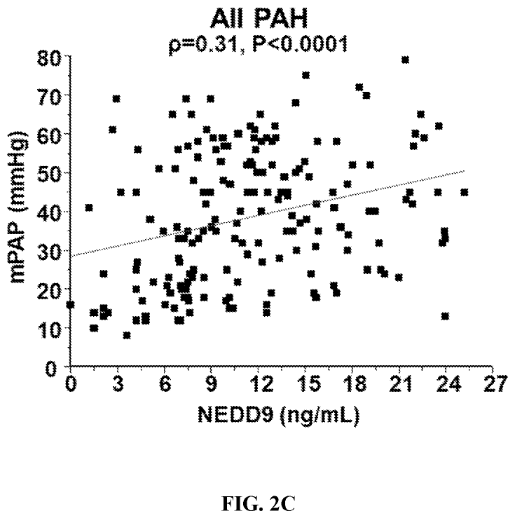

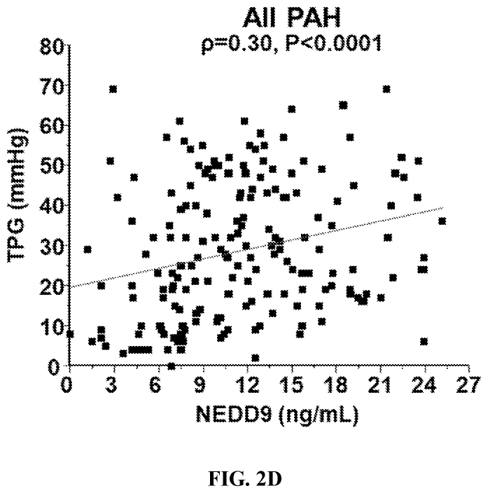

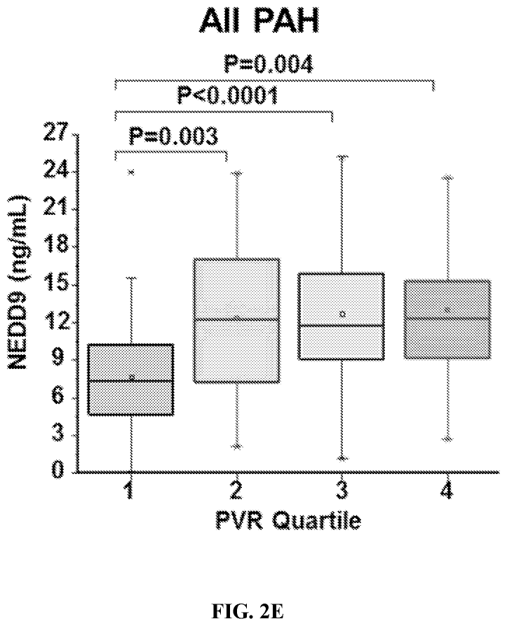

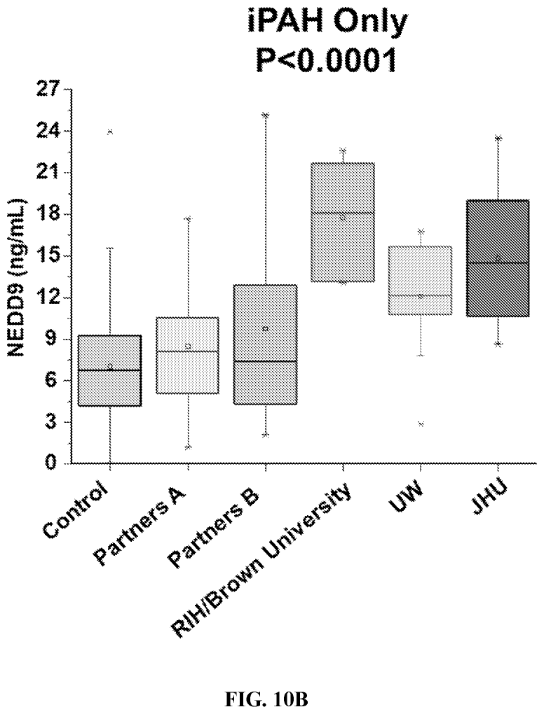

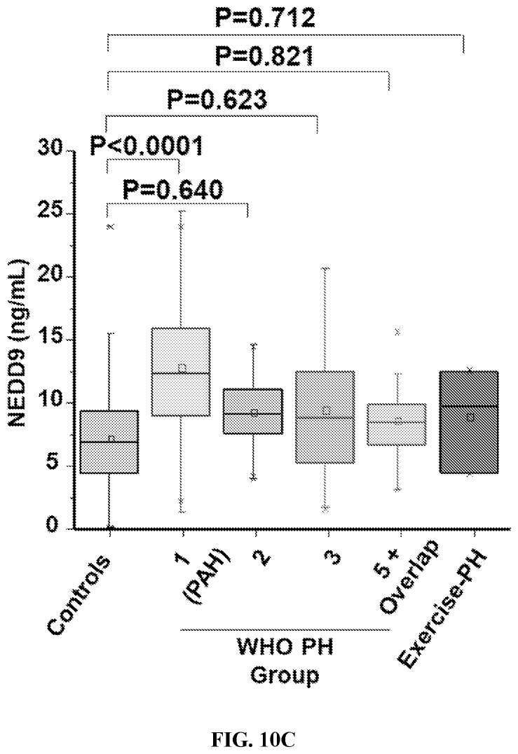

Samples were available for control patients from three centers, and NEDD9 concentration was significantly different across these cohorts (FIG. 10A). Similarly, NEDD9 was different for iPAH patients across all five participating centers (FIG. 9B). Despite this variability, however, NEDD9 was increased overall by 1.82-fold in PAH compared with controls (P<0.0001) (FIG. 2A). No significant difference for NEDD9 was observed by sex (P=0.26), nor was an association evident between NEDD9 and age (ρ=0.09, P=0.20), creatinine concentration (ρ=−0.14, P=0.19), or hemoglobin concentration (ρ=0.23, P=0.053), or when comparing PAH patients for whom PAH-specific medical therapy was vs. was not reported (13.1±4.9 vs. 12.8±6.2 ng/mL, P=0.75).

Next, it was evaluated whether the observed increase in NEDD9 in PAH was driven by a particular PAH subtype. Compared to controls, NEDD9 was increased significantly in iPAH (N=54), CTD-PAH (N=53), CHD-PAH (N=10), Porto-PAH (N=12), and toxin induced-PAH (N=7) (6.7 [4.2-9.6] vs. 12.1±5.6 vs. 13.3±5.5 vs. 16.4±4.0 vs. 9.7±3.0 vs. 13.0±3.3 ng/mL, P<0.0001 by ANOVA) (FIG. 2B). By contrast, plasma NEDD9 discriminated PAH from others forms of pulmonary hypertension, as no significant difference in levels was observed between controls (N=36) and WHO Group 2 PH (N=18), WHO Group 3 PH (N=15), Group 5/mixed phenotype (N=18), and exercise-induced PH (N=3) (FIG. 10B).

### Example 5: NEDD9 and Cardiopulmonary Hemodynamics

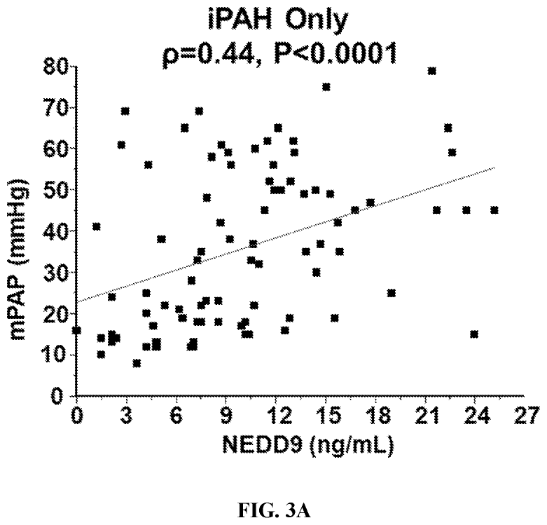

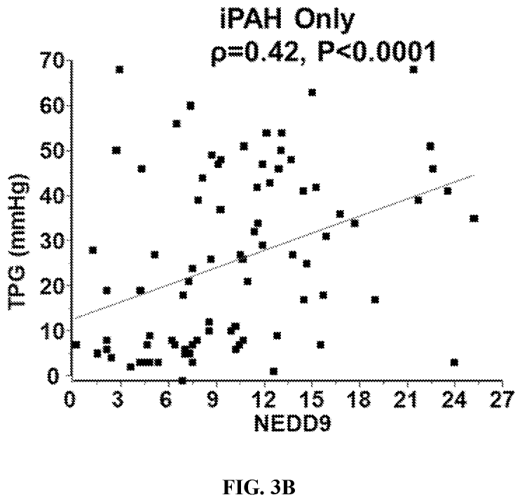

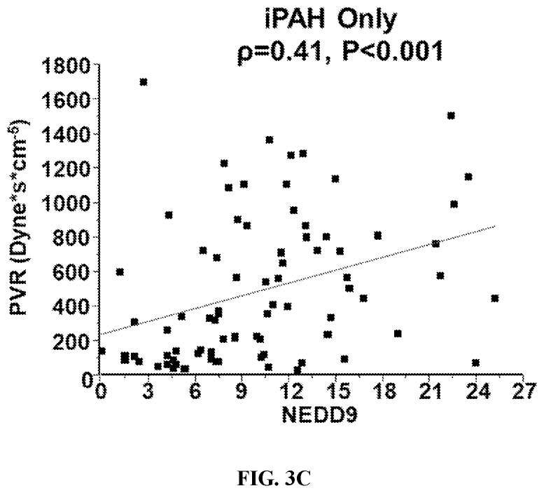

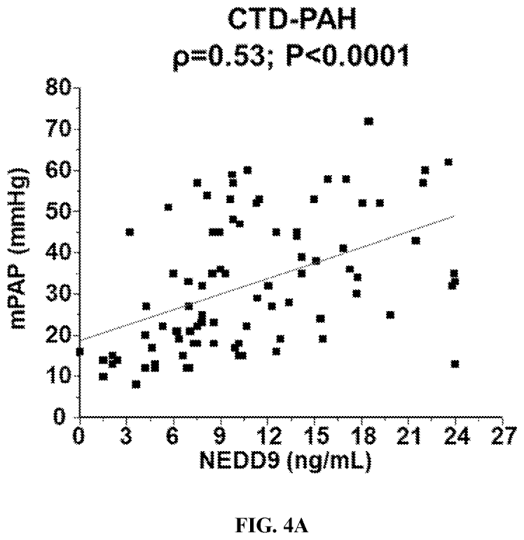

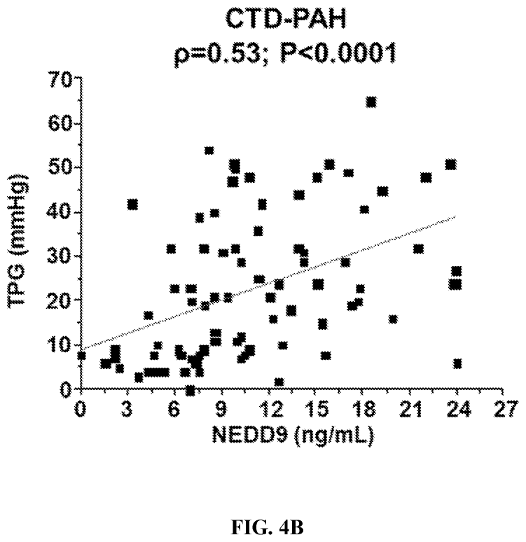

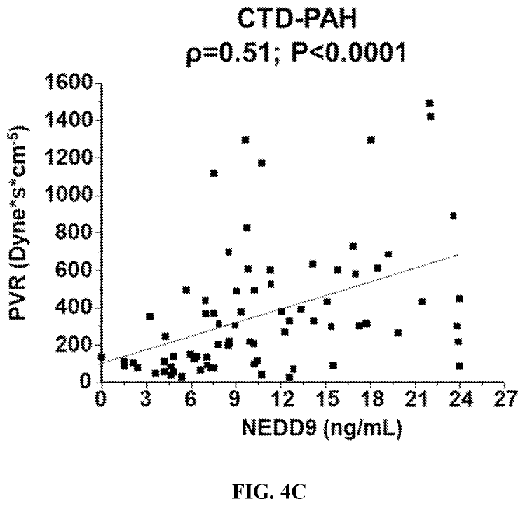

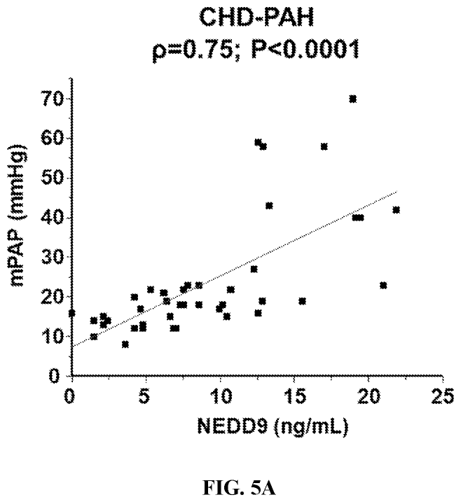

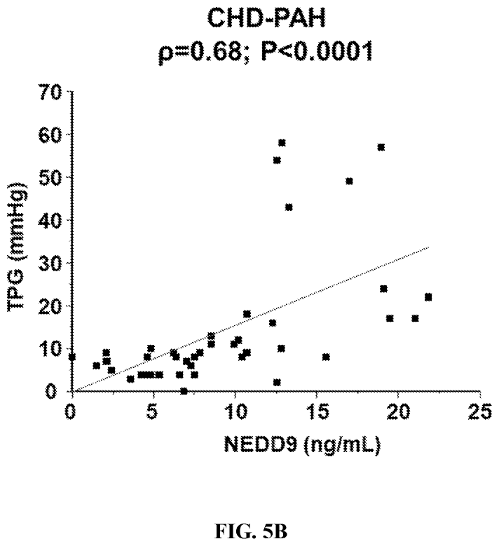

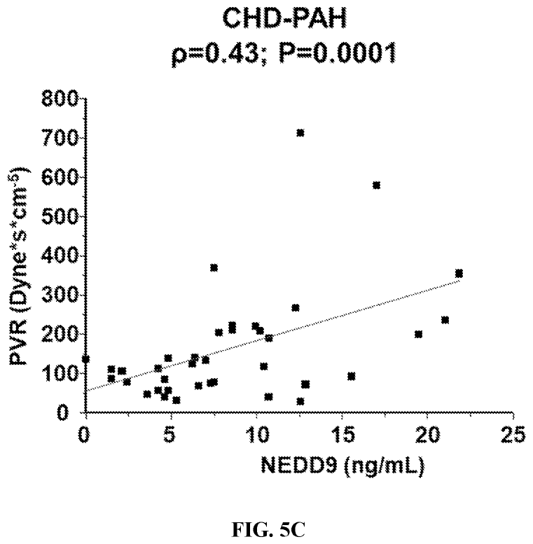

Among controls and PAH patients, there was a borderline moderate, but significant correlation between NEDD9 and mPAP (ρ=0.31, P<0.0001, N=166) and transpulmonary gradient (ρ=0.30, P<0.0001, N=166) (FIGS. 2C-2D). Compared to patients in the lowest PVR quartile, NEDD9 levels were increased in the higher three quartiles (8.2±5.6 vs. 12.4±5.4 vs. 13.1±5.3 vs. 12.3±5.5 ng/mL, P<0.0001 by ANOVA) (FIG. 2E). However, the strength of the association between NEDD9 and mPAP, transpulmonary gradient, and PVR was increased when considering individual PAH subgroups, particularly among iPAH (ρ=0.44, P<0.0001; ρ=0.42, P<0.0001; ρ=0.44, P<0.0001, respectively) (FIGS. 3A-3C), CTD-PAH (ρ=0.53, P<0.0001; ρ=0.53, P<0.0001; ρ=0.51, P<0.0001, respectively) (FIGS. 4A-4C), and CHD-PAH (ρ=0.75, P<0.0001; ρ=0.68, P<0.0001; ρ=0.43, P<0.0001, respectively) (FIGS. 5A-5C). Among these PAH subtypes, sufficient data on 6-minute walk distance were available only for CTD-PAH, which correlated modestly with NEDD9 (ρ=−0.35, P=0.028, N=39). Akin to the overall PAH cohort, NEDD9 did not associate with cardiac output (CO), cardiac index (CI), or PAWP in the full cohort or in any of these subgroups.

These findings indicated that NEDD9 levels in iPAH patients varied by participating center (FIG. 10C), and that NEDD9 most strongly associated with PVR among the hemodynamic measurements analyzed in the full cohort. Therefore, the association between NEDD9 and PVR was used to explore center-specific findings, and a significant increase in PVR per ng/mL change in plasma NEDD9 concentration was observed for the Partners A cohort (N=19) of +28.2 dynes*s*cm⁵ (95% CI: 3.0-53.4, P=0.03) and JHU cohort (N=46) of +39.2 dynes*s*cm⁵ (95% CI: 9.6-68.6, P=0.01) (Tables 16-17).

TABLE 16
Change in PVR as a function of NEDD9 stratified by participating center. The effect increasing NEDD9 on
pulmonary vascular resistance (PVR) was analyzed for controls and PAH patients from all five participating
centers (N = 177). RIH, Rhode Island Hospital; JHU, Johns Hopkins University; CI, confidence interval;
PAH, pulmonary arterial hypertension. PVR is expressed as dynes*s*cm⁵.

| | | | | | | | Age, sex, PAH-subtype | | |
|---|---|---|---|---|---|---|---|---|---|
| | Unadjusted | | | Age-adjusted | | | adjusted | | |
| | ΔPVR | | | ΔPVR | | | ΔPVR | | |
| | per | | | per | | | per | | |
| | 1 ng/mL | 95% | | 1 ng/mL | 95% | | 1 ng/mL | 95% | |
| Center | ΔNEDD9 | CI | P-Value | ΔNEDD9 | CI | P-Value | ΔNEDD9 | CI | P-Value |
| Partners A | 28 | 3.0-53 | 0.03 | 45 | 7.5-82 | 0.03 | 45 | −3.4-94 | 0.06 |
| Partners B | −5.1 | −32-22 | 0.70 | −4.0 | −30-23 | 0.76 | −2.4 | −35-30 | 0.88 |
| RIH/Brown | 3.2 | −21-28 | 0.64 | 4.5 | −62-34 | 0.72 | 0.9 | | 0.68 |
| University | | | | | | | | | |
| University of | −13 | −67-42 | 0.64 | −14 | −21-30 | 0.55 | −8.4 | −50-34 | 0.93 |
| Washington | | | | | | | | | |
| JHU | 39 | 9.6-69 | 0.01 | 39 | 10-68 | 0.01 | 41 | 3.9-77 | 0.03 |

TABLE 17
Summary of data for variables used in the adjusted
model by centers that showed differences in
the relationship between NEDD9 and PVR.

| | Partners A + JHU | Other Centers |
|---|---|---|
| Characteristic | (N = 47) | (N = 130) |
| Age (yr) | 60.0 ± 13.6 | 55.2 ± 15.0 |
| Female (%) | 77 | 57 |
| NEDD9 (ng/mL) | 11.4 ± 5.6 | 13.2 ± 5.2 |
| PVR (dynes*s*cm⁵) | 621 ± 364 | 636 ± 346 |

From Table 16, the Partners A and Johns Hopkins University (JHU) cohorts showed significant differences in the relationship between PVR and NEDD9. This table summarize the differences in age, sex NEDD9 level, and PVR for Partners A + JHU compared to the other participating centers. Data are expressed as mean ± SD, unless otherwise indicated.

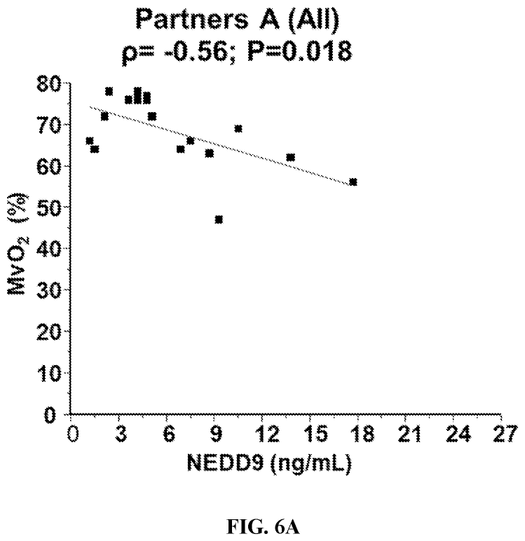

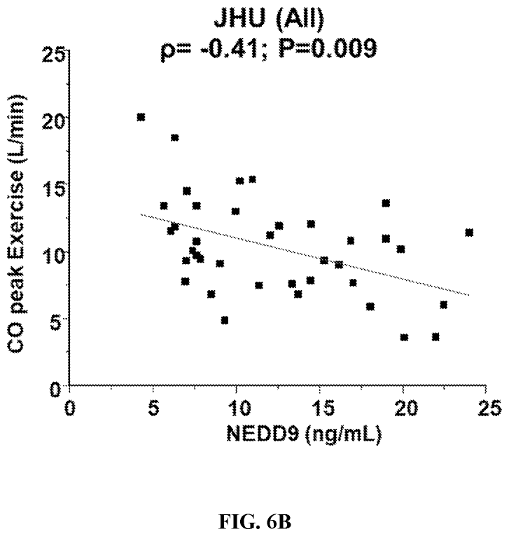

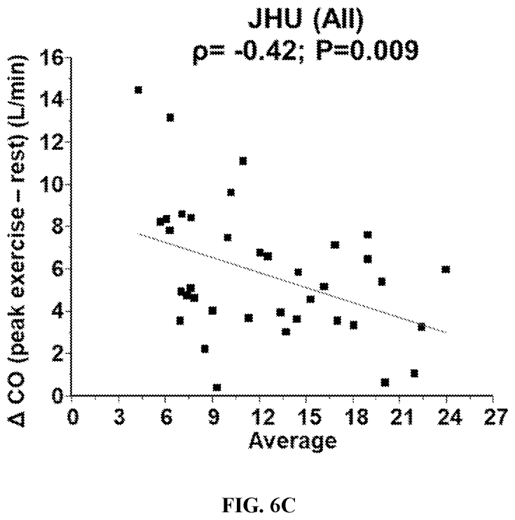

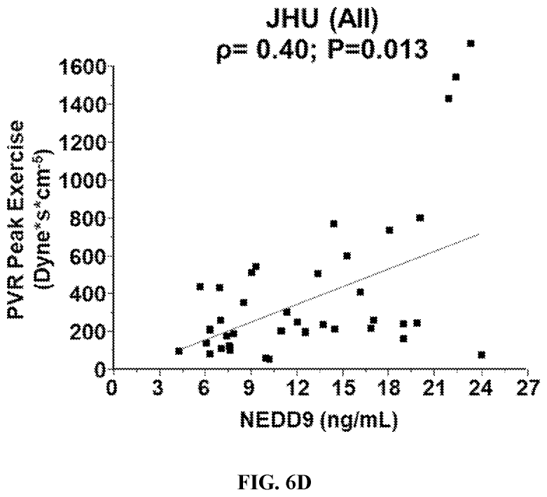

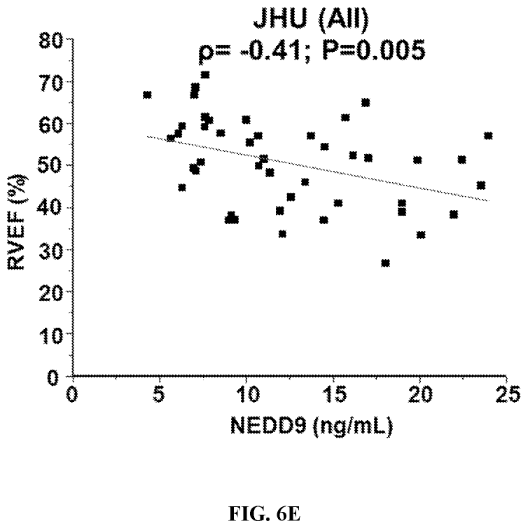

Based on this observation, the Partners A and JHU cohorts were focused on to determine if NEDD9 could inform alternative measurements with prognostic relevance to PAH at these centers specifically. To this end, a modest, inverse correlation between NEDD9 and mixed venous oxyhemoglobin saturation (ρ=−0.56, P=0.018, N=19 Partners A), as well as CO at peak exercise (ρ=−0.41, P=0.009, N=39 JHU), and change in CO from rest to peak exercise (ΔCO) (ρ=−0.42, P=0.009, N=39 JHU) assessed during invasive cardiopulmonary exercise testing was identified. Additionally, NEDD9 correlated moderately with key right ventricular functional and morphologic parameters including ejection fraction (ρ=−0.405, P=0.006, N=45 JHU) and end-systolic volume (ρ=0.313, P=0.036, N=45 JHU) on cardiac MRI (FIGS. 6A-6E).

### Example 6: NT-BNP, NEDD9, and PAH Prognostic Measurements

The N-terminal pro-Brain natriuretic peptide (NT-proBNP) is released from ventricular cardiomyocytes due to pressure-related stretch, is a bona fide prognostic biomarker for PAH patients (Souza et al. N-terminal-pro-brain natriuretic peptide as a haemodynamic marker in idiopathic pulmonary arterial hypertension. Eur Respir J 2005; 25:509-13), and was available for all patients in the JHU cohort (N=46). The association between NT-proBNP and New York Heart Association functional class, 6-minute walk distance, and peak VO₂ was superior compared with NEDD9. However, similar or stronger associations were observed for NEDD9 and peak exercise PVR, CO, ΔCO, as well as resting right ventricular ejection fraction and right ventricular end-systolic volume compared with NT-proBNP. The direction of the association with PAWP at peak exercise diverged for NT-proBNP and NEDD9, in which a positive and inverse correlation of similar magnitude was observed, respectively (Table 18).

TABLE 18
Associations between NT-BNP or NEDD9 with exercise
and cardiac morphological parameters.

| | NT-pro-BNP | | NEDD9 | |
|---|---|---|---|---|
| Variable | ρ coefficient | P-Value | ρ coefficient | P-Value |

Exercise/functional Parameters

| | | | | |
|---|---|---|---|---|
| NYHA FC | 0.305 | 0.039 | 0.185 | 0.218 |
| 6MWD | −0.476 | 0.001 | −0.017 | 0.909 |
| Peak exercise mPAP | 0.317 | 0.043 | 0.124 | 0.440 |
| Peak exercise PAWP | 0.391 | 0.014 | −0.335 | 0.037 |
| Peak exercise CO | −0.542 | 0.001 | −0.413 | 0.010 |
| Delta CO (exercise-rest) | −0.544 | 0.001 | −0.417 | 0.009 |
| Peak exercise PVR | 0.320 | 0.050 | 0.399 | 0.013 |
| Peak VO₂ | −0.384 | 0.023 | −0.194 | 0.265 |
| Ve/VCO₂ | 0.446 | 0.007 | −0.026 | 0.881 |
| Max Watts | −0.518 | 0.001 | −0.007 | 0.967 |

Cardiac morphology/function

| | | | | |
|---|---|---|---|---|
| RV end-diastolic volume | 0.192 | 0.207 | 0.146 | 0.340 |
| RV end-systolic volume | 0.156 | 0.306 | 0.313 | 0.036 |
| RV mass | 0.311 | 0.040 | 0.140 | 0.364 |
| RV ejection fraction | −0.147 | 0.335 | −0.405 | 0.006 |

Invasive exercise and cardiac structural and functional data acquired by cardiac magnetic resonance imaging were analyzed for all patients in the Johns Hopkins University cohort (N = 46). Values for NT-BNP and NEDD9 were not normally distributed in this population; therefore, data are presented as Spearman correlation coefficient for each association. Statistically significant associations for NEDD9 that were stronger than NT-BNP, which is the current pulmonary vascular disease biomarker standard, are bolded. NYHA FC, New York Heart Association functional class; 6MWD, 6-minute walk distance; mPAP, mean pulmonary artery pressure; PAWP, pulmonary artery wedge pressure; CO, cardiac output; PVR, pulmonary vascular resistance; VO₂, volume of oxygen consumption; RV, right ventricle; LV, left ventricle.

### Example 7: Plasma NEDD9 and Clinical Outcome

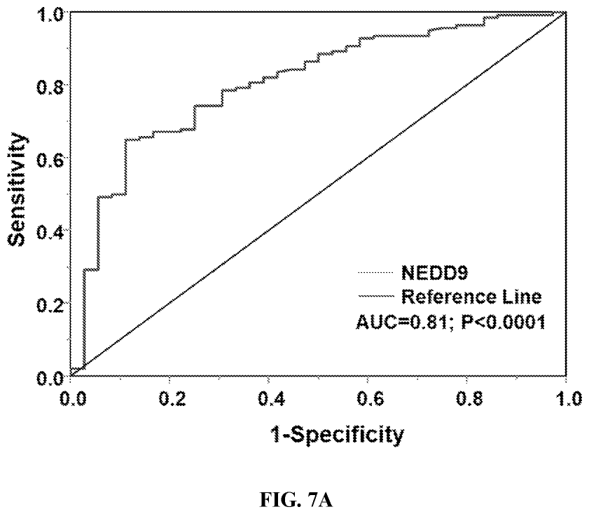

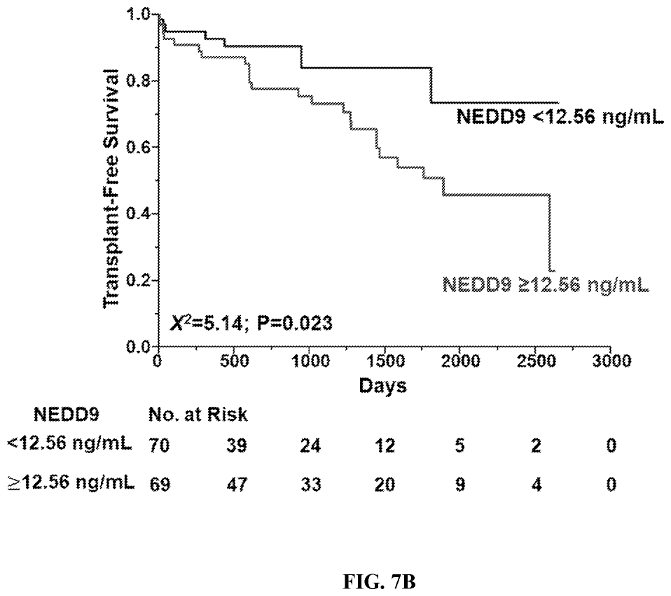

The AUC for predicting PAH diagnosis from plasma NEDD9 was 0.81 (P<0.0001) (FIG. 7A). The event free curve for the combined end-point of lung transplant or all-cause mortality is presented in FIG. 7B. The PAH cohort was followed for a median of 874 d. Overall, 21.5% of the cohort had an event, including N=28 deaths and N=6 lung transplants. The 1-year and 3-year event rates were 8.9% and 15.8%, respectively. The lung transplant-free survival hazard ratio (HR) after adjusting for age, sex, referral center, and PAH subtype when modeling plasma NEDD9 as a continuous variable was 1.12 (95% CI 1.02-1.22, p=0.01) and 1.75 (95% CI 1.12-2.73, p=0.01) per 1 ng/mL and 5 ng/mL increase, respectively. Compared to patients with a plasma NEDD9 level <12.56 ng/mL, the HR for event-free survival among patients with NEDD9≥12.56 ng/mL was 3.28 (95% CI 1.15-9.37, P=0.03).

### Example 8: NEDD9 is Increase in CTEPH, ARDS, and COVID

NEDD9 concentrations in plasma specimens from patients with chronic thromboembolic pulmonary hypertension (CTEPH) (N=27), and plasma specimens from N=7 age- and sex-matched healthy controls (N=7) were analyzed by ELISA. Compared to controls, plasma NEDD9 was increased significantly in CTEPH patients compared with age- and sex-matched healthy controls (3.7±0.6 vs. 10.6±3.9 ng/mL, P<0.001).

It was also observed that, compared to controls, plasma NEDD9 levels are increased significantly in patients with typical acute respiratory distress syndrome (ARDS)(N=9), COVID-19 (N=20), and COVID-19+ARDS (N=20) (3.7±0.6 vs. 7.9±4.1 vs. 10.0±5.0 vs. 8.5±4.1 ng/mL, P<0.05).

In additional studies, the mean plasma NEDD9 (ng/ml) was detected in samples from patients with these different respiratory pathophenotypes to yield the following results: Healthy Controls (N=7) 5.5+/−1.0 ng/mL; Non-COVID ARDS (N=4) 10.2+/−3.7 ng/mL; COVID+ARDS (N=19) 9.5+/−3.7 ng/mL; and COVID without ARDS (N=17) 10.6+/−3.4 ng/mL.

Taken together, these results demonstrate that an elevated level of NEDD9 can be detected in plasma samples from patients having CTEPH, ARDS, and COVID.

### OTHER EMBODIMENTS

All of the features disclosed in this specification can be combined in any combination. Each feature disclosed in this specification can be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments can be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases can encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for analyzing a sample, the method comprising:
providing a sample from a subject, and
detecting neural precursor cell expressed developmentally down-regulated protein 9 (NEDD9) in the sample.

2. The method of claim 1, wherein detecting NEDD9 in the sample comprises detecting a level of NEDD9 protein in the sample.

3. The method of claim 2, wherein the level of NEDD9 protein is detected by an immunoassay.

4. The method of claim 3, wherein the immunoassay is an enzyme-linked immunosorbent assay (ELISA).

5. The method of claim 1, wherein detecting NEDD9 comprises detecting a level of NEDD9 nucleic acid.

6. The method of claim 1, wherein the sample is a plasma sample.

7. The method of claim 1, wherein the sample is obtained from a subject having or suspected of having pulmonary hypertension (PH).

8. The method of claim 7, wherein the pulmonary hypertension (PH) is selected from the group consisting of pulmonary arterial hypertension (PAH), chronic thromboembolic pulmonary hypertension (CTEPH), and pulmonary hypertension (PH) due to acute respiratory distress syndrome (ARDS).

9. The method of claim 1, further comprising administering the subject a treatment for pulmonary hypertension (PH).

10. The method of claim 1, wherein the subject is a human patient or a non-human animal.

11. A method of diagnosing a subject as having pulmonary hypertension (PH), the method comprising:
providing a sample from the subject,
detecting a level of NEDD9 in the sample; and
comparing the level of NEDD9 in the sample to a reference level, wherein the presence of a level of NEDD9 in the sample that is above the reference level indicates that the subject has PH.

12. The method of claim 11, wherein detecting NEDD9 in the sample comprises detecting a level of NEDD9 protein in the sample.

13. The method of claim 12, wherein the level of NEDD9 protein is detected by an immunoassay.

14. The method of claim 13, wherein the immunoassay is an enzyme-linked immunosorbent assay (ELISA).

15. The method of claim 11, wherein detecting NEDD9 comprises detecting a level of NEDD9 nucleic acid.

16. The method of claim 11, wherein the sample is a plasma sample.

17. The method of claim 11, wherein the sample is obtained from a subject having or suspected of having pulmonary hypertension (PH).

18. The method of claim 17, wherein the pulmonary hypertension (PH) is selected from the group consisting of pulmonary arterial hypertension (PAH), chronic thromboembolic pulmonary hypertension (CTEPH), and pulmonary hypertension (PH) due to acute respiratory distress syndrome (ARDS).

19. The method of claim 11, further comprising administering the subject a treatment for pulmonary hypertension (PH).

20. The method of claim 11, wherein the subject is a human patient or a non-human animal.
